(19) United States
(12) Patent Application Publication
(10) Pub. No.: US 2011/0236937 A1
(43) Pub. Date: Sep. 29, 2011

(54) Multi-Stage Fermenter Nutrient Feeding

(75) Inventors: Aaron Douglas Smith, Bryan, TX (US); Mark T. Holtzapple, College Station, TX (US)

(73) Assignee: THE TEXAS A&M UNIVERSITY SYSTEM, College Station, TX (US)

(21) Appl. No.: 13/071,059

(22) Filed: Mar. 24, 2011

Related U.S. Application Data
(60) Provisional application No. 61/317,125, filed on Mar. 24, 2010.

(51) Int. Cl. C12P 7/40; C12P 1/00; C12M 1/34; C12M 1/36

(52) U.S. Cl. 435/136; 435/41; 435/287.1; 435/286.5

(57) ABSTRACT

A method for operating a fermenter system. In one instances, the method comprises flowing biomass and liquid in opposite directions through a fermenter train comprising a plurality of fermenters, and introducing a nutrient to any of the plurality of fermenters to optimize the production carboxylate products in the fermenter system.

### CROSS-REFERENCE TO RELATED APPLICATIONS

This application claims benefit under 35 USC §119 of U.S. provisional application No. 61/317,125 filed Mar. 24, 2010, entitled “Fermenter Nutrient Feeding” which is hereby incorporated herein by reference in its entirety for all purposes.

### STATEMENT REGARDING FEDERALLY SPONSORED RESEARCH OR DEVELOPMENT

Not applicable.

### BACKGROUND

1. Field of the Invention

This invention relates to the fermenting of biomass, specifically to the arrangement and operation of a biomass fermentation system.

2. Background of the Invention

The production of liquid fuels, chemicals, and solvents from carboxylic acids provides a commercial alternative to conventional petroleum distillation. The carboxylic acids may be produced by anaerobic fermentation of biomass, using microorganisms derived from animal rumen, insects, compost, sediment, and other environments with anaerobic decomposition of biomass. During the anaerobic fermentation the wetted biomass is digested into a fermentation broth including the carboxylic acid products.

Generally, the fermentation broth includes carbohydrate-rich and nutrient-rich components derived from the biomass. Without limitation by theory, the carbohydrate-rich fermentation broth components are the energy and carbon sources for the fermentation, while nutrient rich sources include all other compounds that are essential to life. Non-limiting examples of carbohydrate-rich components of the fermentation broth may include, cellulose, hemicellulose, lignin, starch, other carbohydrates derived from sugarcane bagasse, corn stover, wood, landscaping waste, and municipal solid waste. Nutrient-rich components of the fermentation broth include proteins, polypeptides, amino acids, nucleic acids, fats, minerals, salts, ions, metals, phosphorous, sulfur, other elements and components essential to life, without limitation.

During fermentation, the broth's chemical composition is altered, and the reactivity of carbohydrate-rich components decreases as they are digested. Additionally, buffers are added to the broth to help maintain a preferred pH for the microorganisms. Otherwise, the pH may inhibit further reaction, thereby reducing acid production. Further in liquid-solid counter-current fermentations, certain soluble nutrient-rich components are prematurely removed from the fermenter or fermenters with the liquid stream. The premature removal of the nutrient-rich components may deprive the microorganisms in the fermenter(s) and reduce or inhibit continued digestion and acid production.

As such, there is a potential commercial demand for a fermenter apparatus and method of operation that can control optimal nutrient concentrations such that carboxylic acid production for chemical, solvent, and liquid fuel synthesis.

### BRIEF SUMMARY

A method for fermenting biomass comprising, fermenting biomass in a first fermenter to form digested biomass and a first fermentation broth, introducing the digested biomass from the first fermenter to a second fermenter having a second fermentation broth, and introducing a nutrient to at least one of the fermenters. In embodiments, the method comprises detecting a property of the fermentation broth in each of the fermenters; and analyzing the property of the fermentation broth in each of the fermenters prior to introducing a nutrient. Also, the method comprises measuring a concentration of the nutrient in the first fermenter broth and the second fermenter broth, and the analyzing comprises determining the difference in the concentration of the nutrient in the first fermenter broth and the second fermenter broth. The method comprising determining the difference in the concentration of a fermentation product in the first fermenter broth and the second fermentation broth for detecting and analyzing. The method as above, wherein the fermentation product comprises a carboxylate product, the nutrient comprises undigested biomass, and wherein the nutrient comprises essential components for life processes.

A method for fermenting biomass comprising, fermenting biomass in a first fermenter to form digested biomass and a first fermentation broth, introducing the digested biomass from the first fermenter to a second fermenter having a second fermentation broth, and introducing a carbon source to at least one of the fermenters. The method comprises detecting a property of the fermentation broth in each of the fermenters, and analyzing the property of the fermentation broth in each of the fermenters prior to introducing a carbon source. Also the method comprises measuring a concentration of the carbon source in the first fermenter broth and the second fermenter broth, and the analyzing comprises determining the difference in the concentration of the carbon source in the first fermenter broth and the second fermenter broth. The method comprising determining the difference in the concentration of a fermentation product in the first fermenter broth and the second fermentation broth. The method as above, wherein the fermentation product comprises a carboxylate product, the carbon source comprises undigested biomass, and wherein the carbon source comprises any biologically available carbon source for essential life processes.

A method for fermenting biomass comprising, fermenting biomass in a first fermenter to form digested biomass and a first fermentation broth, introducing the digested biomass from the first fermenter to a second fermenter having a second fermentation broth, and introducing a nutrient and a carbon source to at least one of the fermenters. The method comprising detecting at least one property of the fermentation broth in each of the fermenters, and analyzing at least one property of the fermentation broth in each of the fermenters, prior to introducing a nutrient and a carbon source. Also the method comprises measuring a concentration of the fermentation product in the first fermenter broth and the second fermenter broth, and the analyzing comprises determining the difference in the concentration of fermentation product in the first fermenter broth and the second fermenter broth. The method comprising determining the difference in the concentration of the nutrient in the first fermenter broth and the second fermentation broth and determining the difference in the concentration of the carbon source in the first fermenter broth and the second fermentation broth. In instances, the method comprises determining the difference in ratio of the concentration of the nutrient to the concentration of the carbon source in the first fermenter broth and the second fermentation broth. According to another embodiment of the method the nutrient comprises introducing undigested biomass. The method comprising introducing the nutrient to the first fermenter and introducing the carbon source to the second fermenter, introducing the nutrient to the second fermenter and introducing the carbon source to the first fermenter, or introducing the nutrient and the carbon source at a predetermined ratio.

A method for fermenting biomass comprising, fermenting a first biomass in a first fermenter to form a first digested biomass and a first fermentation broth, fermenting a second biomass in a second fermenter to form a second digested biomass and a second fermentation broth, fermenting a third biomass in a third fermenter to form third digested biomass and a third fermentation broth, detecting at least one property of each of the fermentation broths for analysis, and introducing the first digested biomass to the second fermentation broth in the second fermenter, introducing the first fermentation broth to the third digested biomass in the third fermenter; and introducing the third fermentation broth to the second digested biomass. Further, the method, wherein the first biomass comprises undigested biomass and the second biomass and third biomass comprise at least partially digested biomass. Further, the method comprises comparing the least one detected property of each fermentation broths against each other and against a predetermined optimization of the at least one detected property and determining which fermentation broth to introduce to which digested biomass. The at least one property may comprise one chosen from the group consisting of pH, nutrient concentration, carbon source concentration, nutrient concentration to carbon concentration ratio, and combinations thereof.

A fermenter system comprising, a plurality of fermenters, having a first fermenter, a last fermenter, and at least one intermediate fermenter, wherein the first fermenter comprises the inlet for biomass, and the last fermenter comprises the inlet for fermentation broth, a plurality of conduits disposed between each of the plurality of the fermenters, a sensor system, having a sensor positioned in each of the plurality of fermenters, and a nutrient supply, fluidly connected with each of the plurality of fermenters. Also, the system comprises a first portion of the plurality of conduits configured to convey biomass between each of the plurality of fermenters, and a second portion of the plurality of conduits configured to convey fermentation broth between each of the plurality of fermenters. The system comprising a control system, wherein in response to the sensor system, the control system is configured to control flow through the plurality of conduits and the nutrient supply and wherein the nutrient supply comprises at least one selected from the group consisting of: undigested biomass, a nutrient-rich supply, a carbon-rich supply, and combinations thereof.

The foregoing has outlined rather broadly the features and technical advantages of the invention in order that the detailed description of the invention that follows may be better understood. It should also be realized by those skilled in the art that equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates a schematic according to one embodiment of the present disclosure

FIG. 2 illustrates a block flow diagram according to one embodiment of the present disclosure.

FIG. 3 illustrates a block flow diagram of the MixAlco process.

FIG. 4 illustrates the conversion of biomass according to another embodiment of the disclosure.

FIG. 5 illustrates a four-stage countercurrent fermentation train with digestion and dilution gradients, according to another embodiment of the disclosure.

FIG. 6 illustrates an alternative configuration for a countercurrent fermentation train.

FIG. 7 illustrates the nutrient loading pattern for Trains 1, 2, 3, 4, and P with the amount of wet chicken manure (CM; on a dry basis) added to each fermenter.

FIG. 8 illustrates the carbon-nitrogen ratio profiles for each train. Carbon contributed by organic acid was excluded.

FIG. 9 illustrates the productivity profiles for each train, representing the composite productivity of the train.

FIG. 10 illustrates the correlation between productivity and C/N ratio for individual fermenter and train.

FIG. 11 illustrates the correlation between productivity and C/N ratio for individual fermenter and train.

FIG. 12 illustrates the total acid concentration and acetic acid equivalence concentration plots.

FIG. 13 illustrates the comparison of yield values for each train.

FIG. 14 illustrates a segregated nitrogen input countercurrent fermentation train.

FIG. 15 illustrates an equation matrix for the total and moisture mass balance in a four-stage countercurrent fermenter.

FIG. 16 illustrates an equation matrix for the nitrogen mass balance in a four-stage countercurrent fermenter.

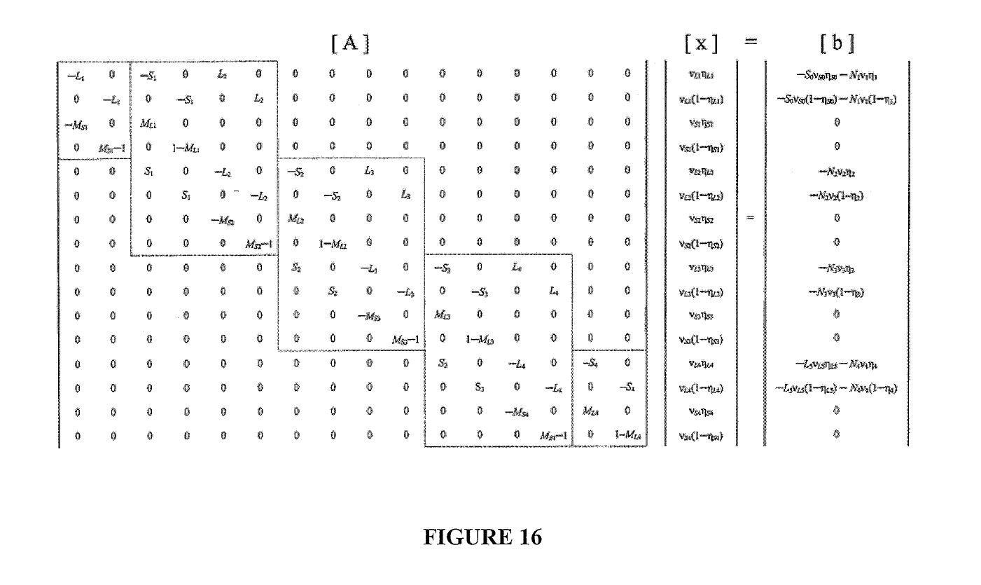

FIG. 17 illustrates the measured soluble nitrogen fraction.

FIG. 18 illustrates the predicted and measured nitrogen concentration in each train.

FIG. 19 illustrates the predicted and measured C/N ratio.

FIG. 20 illustrates the absolute error.

### DETAILED DESCRIPTION OF THE PREFERRED EMBODIMENTS

OVERVIEW: The production of chemicals and fuels from biomass may be mediated through anaerobic, mixed-acid fermentation. Mixed-acid fermentation produces carboxylic acids and salts, hereinafter carboxylate products. The carboxylate products may be further processed into chemicals and fuels. Using fermentation as a carboxylate source is economically favorable with high carboxylate product yields in the fermentation broth. However, varied biomass compositions result in altered or unpredictable carboxylate yields, making the fermentation process a rate and yield limiting step in the production of chemicals and fuels.

The biomass comprises a nutrient-rich portion and a carbon-rich portion. The nutrient-rich portion is the primary source of other essential components for a microorganism's life processes. In embodiments, nutrients may include, elemental or atomic matter, metals, alloys, minerals, biomolecules such as RNA/DNA, fats, co-factors, amino acids, proteins, and combinations thereof, without limitation. Additionally, the nutrient-rich portion comprises any source of nitrogen. Exemplary sources of nitrogen may include without limitation, diatomic nitrogen (i.e. nitrogen gas), nitrates, nitrides, nitrites, azides, ammonia, ammonium, urea, uric acid, and combinations thereof. Nutrient-rich biomass may be waste sludge, slaughter house waste, farm waste, roadkill, municipal waste, or other decaying animal matter.

The carbon-rich portion generally comprises any biologically available carbon based molecule capable of being metabolized for energy, molecular synthesis, or combinations thereof by a microorganism. Without limitation by theory, the carbon rich portion of the biomass comprises a carbon source for essential microorganism life processes and energy. More specifically, the carbon-rich portion of the biomass comprises carbohydrate carbon sources, such as but not limited to cellulose, hemicellulose, starch, polymeric sugars, oligomers, monomers, and combinations thereof. Without limitation, the carbon-rich portion of biomass generally comprises plant matter, such as yard waste, farm waste, landscape waste, corn stovers, bagasse, paper waste, and other decaying plant materials

During fermentation anaerobe access to the nutrient-portion and carbon-portion of the biomass changes with increased digestion and decreased biomass reactivity. The nutrient concentrations and the carbon concentrations may decrease significantly with increased residence time. Additionally, with increased residence time, the relative proportions of nutrients to carbon in the fermentation broth fluctuate significantly, resulting in lower product yields. The anaerobes (anaerobic microorganisms) in the fermenter have a variety of nutrient concentrations, carbon concentrations, and/or nutrient-carbon ratios that optimize the production of carboxylates and maximize the efficiency of the fermentation to products. Additionally, under certain conditions in the fermentation media or fermentation broth, the anaerobes may produce excessive concentrations of the carboxylates. The production and accumulation of carboxylate products in the fermentation broth inhibits further fermentation by lowering the pH, in some instances to below about pH 4.8. Although buffers may be utilized to counter this effect, the digestion of the biomass to carboxylic acids and salts via anaerobic pathways slows and potentially stops. In order to avoid these unwanted decreases in fermentation efficiency alternate protocols to batch processing are needed.

A counter-current fermenter system increases the fermentation efficiency and carboxylate yields, but does not provide a solution for the nutrient and carbon concentration fluctuation. A counter-current fermenter system comprises a plurality of connected fermenters that exchange biomass and fermentation broth in opposite flow directions. In certain circumstances, the fermenters are arranged such that each has a progressively older biomass loading; that is the biomass in each fermenter has had a longer fermentation period since inoculation. The partially digested biomass may be moved sequentially through the fermenters. Fermentation broth, comprising a significant proportion of water is circulated in the opposite direction. The most dilute fermentation broth is used for biomass that has been fermenting the longest. Without limitation by theory, the dilute fermentation broth removes inhibition discussed previously to the continued digestion of the most degraded biomass, therefore increasing the yield and efficiency. As the fermentation broth becomes increasingly concentrated, it is moved to newer or fresher sources of biomass, which containing increased concentrations of nutrients and carbon sources. The most concentrated fermentation broth is used for the fermentation of fresh biomass, immediately prior to product recover.

MULTI-STAGE FERMENTATION: The present disclosure relates to the fermentation systems and methods that are supplemented by cross-flow or targeted addition of material to the fermenters. In certain instances, the fermentation systems may be concurrent flow or cross-flow fermentation systems. The method may be considered to alter the fermenter steps from time interval based fermentation steps to fermentation broth product concentration stages. More specifically, the present disclosure relates to a method of feeding nutrients and carbon sources to individual fermenters at different stages of fermentation, in order to maximize the carboxylate production. Further, the process comprises monitoring the concentration of certain predetermined chemicals or molecules, hereinafter nutrients, in the fermentation broth of each fermenter. In instances, the process includes introducing a supplemental amount of the predetermined nutrients, monitoring the production of carboxylates in the fermenters, comparing the production of carboxylates to a predetermined fermentation model, and repeating the addition of those predetermined nutrient in order to maximize the production of carboxylates. Alternatively, the addition of nutrients may comprise the addition of undigested biomass to a partially digested fermentation broth in order to increase carboxylate product synthesis. The addition of undigested biomass provides impetus for the re-initiation of high yield carboxylic acid production and fermentation. In further alternate methods, the fermentation broth and biomass are moved between fermenters in the fermenter system based on fermenter broth carboxylate product concentrations, nutrient concentrations, carbon-source concentrations, carboxylate product inhibitor concentrations, or any combination thereof.

In the following discussion, nutrients may be added to any of the fermenter stages in order to maximize carboxylate production. The nutrients may be any nutrient without limitation by the following discussion, for example, the nutrients may by organic nutrient, such as proteins and amino acids, or any inorganic nutrient such as minerals and salts, without limitation. The nutrients may comprise un-isolated or raw nutrients, isolated nutrients, partially isolated nutrients, purified nutrients, partially purified nutrients, biochemically similar nutrients, and any combination of thereof. Additionally, due to the compositional differences in the varied sources of biomass, any nutrient may need to be supplied continuously to the fermenters, biomass, fermentation broth, or combinations thereof in order to maximize carboxylate production.

In the present disclosure, the method of directing the fermentation broth through the fermenters based on the concentration of carboxylate product, nutrients, carbon sources, or combinations thereof provides a method to achieve higher concentrations of carboxylates prior to separation for processing while maintaining a carboxylate concentration below the threshold tolerance for the microorganisms and in the presence of the highest concentration of available carbon sources. Additionally, the direction of biomass and fermentation broth flow through the fermentation system provides a means to maintain an elevated carboxylate production through multiple intra-system biomass and fermentation broth transfers. As such, the term “stage” may refer to the progression of fermentation in the fermenters rather than a sequential progression of biomass through the system.

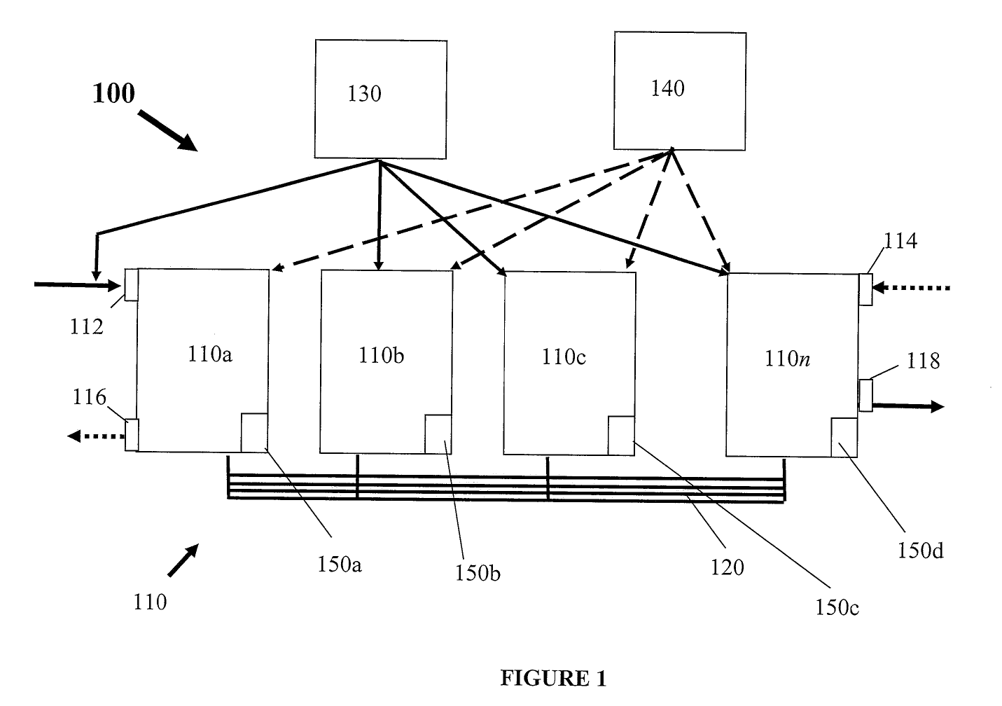

FERMENTERS: Referring to FIG. 1, in embodiments, the present disclosure relates to a plurality of interconnected fermenters 110 in a fermentation system 100. Each of the fermenters 110 may have any configuration for retaining aqueous slurry of biomass. Additionally, the fermenters 110 may be any fermenter known to a skilled artisan, including but not limited to, pit fermenters, warehouse fermenters, tank fermenters, trickling fermenters, rotating drum fermenters, or any other vessel suitable for fermenting biomass. The fermenters may be constructed of any suitable material without limitation and as discussed herein, the fermenters include all associated peripheral equipment such as, pipes, pumps, valves, filters, vents, drains, apparatuses, and devices to facilitate fermentation. Exemplary fermenters include U.S. Pat. No. 5,874,263 U.S. Pat. No. 5,962,307, U.S. Pat. No. 6,395,926, U.S. patent application Ser. No. 12/555,184, and U.S. patent application Ser. No. 12/629,285 without limitation.

Additionally, the fermenters 110 may include a means to circulate the fermentation broth throughout the biomass. The fermenters 110 may be configured to release, capture, or recapture gas produced from the fermentation reactions for recirculation. In certain configurations, the fermenters 110 have inlets such as inlet 112 to the first fermenter 110a, or inlet 114 to the last fermenter 110n. Inlets to the fermenters may be used for the addition of gases, liquids, or solids including chemicals, nutrients, biomass, buffers, fermentation broth, or water, without limitation. Further, the tormenters 110 may have outlets, such as outlet 116 from the first fermenter 110a or outlet 118 from to the last fermenter 110n. Outlets from the fermenters may be used for removing gases, liquids, or solids, including digested biomass waste, fermentation broth, carboxylate products, buffer salts, cellular debris, and water, without limitation. Alternatively, the inlets and outlets on each fermenter may comprise an inlet or outlet from the fermenter system 100.

FERMENTER SYSTEM COMPONENTS: The fermenter system 100 comprises a plurality of fermenters 110 or fermentation stages. The fermenters 110 may be considered a first fermenter 110a, second fermenter 110b, third fermenter 110c, etc. to a last fermenter 110n, without limitation. The last fermenter may also be termed an n^{th} fermenter, wherein the value of n is any positive integer; in embodiments n is between about 2 fermenters and about 10 fermenters; alternatively, n is between about 3 fermenters and about 8 fermenters; and in certain instances, n is between about 4 fermenters and 6 fermenters in the fermenter system. Alternatively, the fermenter system may have any number of fermenters 110 to produce carboxylate products

In embodiments, the fermenter system 100 comprises a plurality of conduits 120. As may be understood by a skilled artisan the term conduit or conduits 120, refers to any means configured to convey or communicate materials including gases, liquids, solids, and combinations thereof from one location to another within the fermenter system 100. Additionally, as discussed herein conduits 120 may include fermenter inlets, fermenter outlets, pumps, valves, filters, vents, and all other devices or apparatus that participate or aid in material communication between the fermenters. In certain embodiments, the fermenter system 100 comprises solids conduits and fluid conduits for separate transport of solids and fluids, respectively.

In certain embodiments, the fermenter system comprises a plurality of carbon-rich 130 sources and nutrient-rich 140 sources. The carbon-rich 130 sources and nutrient-rich 140 sources comprise a continuous or discontinuous feedstream from other processes, stored materials, or commercially available materials. In exemplary embodiments, carbon-rich 130 sources comprise carbohydrate-rich components, such as but not limited to sugarcane, bagasse, corn stover, wood, municipal solid waste, landscape and construction debris. The carbohydrate components in the carbon sources 130 include cellulose, hemicellulose, lignin, starch, sugar, pectin, and other carbohydrate monomers, oligomers, and polymers, without limitation. In certain instances the carbon sources comprises a carbon source for essential microorganism life processes and energy.

In exemplary embodiments, nutrient-rich sources 140 comprise biomolecular-components such as food scraps, sewage sludge, manure, roadkill, and slaughterhouse waste, without limitation. The biomolecular components include proteins, amino acids, polypeptides, DNA, RNA, fats, lipids, vitamins, co-factors, and salts as non-limiting examples. Additionally, nutrient-rich sources 140 may comprise minerals, metals, electrolytes, ions, salts, and other inorganic compounds derived from certain chemical processes. Further, the nutrient rich sources 140 comprise nutrients with a high nitrogen content, for example without limitation diatomic nitrogen (i.e. nitrogen gas), nitrates, nitrides, nitrites, azides, ammonia, ammonium, urea, uric acid, and combinations thereof. Alternatively, the carbon-rich 130 and nutrient-rich sources 140 may be purified sources that have been industrially or commercially produced as side products or as reactants for other processes.

In embodiments, the fermenter system 100 comprises a plurality of sensors 150. The sensors 150 are any means configurable to detect any properties of the biomass and fermentation broth. The sensors 150 may detect the physical or chemical properties, such as but not limited to temperature, pH, suspended solids, microorganism population, microorganism metabolism, and the concentration of nutrient-rich materials, carbon-rich materials, carboxylate products, and buffer concentration. In certain instances, the sensors 150 may be any device capable of detecting a physical, chemical, or biological property of the biomass and fermentation broth. Alternatively, the sensors 150 may comprise an apparatus or device configured to withdraw a sample of the biomass or fermentation broth from each fermenter 110a, 110b, etc for human analysis, for example in a laboratory. In certain instances, the fermenter system 100 comprises a laboratory for analysis of samples including but not limited to the biomass, fermenter broth, and gases released during fermentation. In further instances, a sensor 150 may comprise a nutrient or carbon-source control system. The sensor 150 is configured to adjust the volume or mass of the nutrient or carbon-source feed into one or more of the plurality of fermenters when the detected concentration is outside of a predetermined range.

FERMENTER SYSTEM CONFIGURATION: In embodiments, each fermenter in the fermenter system is connected to at least one additional fermenter either directly or indirectly by a conduit 120. In certain embodiments, the fermenter system 100 is configured for biomass-fermentation broth counter-current. Alternatively, each fermenter 110a, 110b, etc is connected with all other fermenters in the fermenter system 100, either directly or indirectly. In further embodiments, each fermenter 110a, 110b, etc is connected to at least one carbon-rich source 130 and at least one nutrient rich source 140 either directly, or indirectly. Each fermenter 110a, 100b, 110c, etc, has an associated sensor 150a, 150b, etc.

The fermenter system 100 is configured to receive a first portion of fresh or undigested biomass at an inlet 112 disposed on a single fermenter, hereinafter the first fermenter 110a. The fermenter system 100 is configured to withdraw, for example via an outlet 118, the partially digested, carbon-depleted, or waste biomass at a separate fermenter, hereinafter the last fermenter 110n. The fermenter system 100 is configured to move the biomass through the intervening fermenters 110b, 110c, etc, at predetermined intervals. In a counter-current configuration, the fermenter system 100 is arranged to receive a portion of fermentation broth at the last fermenter 110n inlet 114 and withdraw the fermentation broth, comprising the carboxylate products at the first fermenter 110a outlet 116. In a concurrent configuration, the fermenter system 100 is arranged to receive a portion of fermentation broth at the first fermenter 110a inlet 112 and withdraw the fermentation broth, comprising the carboxylate products at the last fermenter 110n outlet 118. The fermenter system 100 is configured to move the fermentation broth through the intervening fermenters 110b, 110c, etc, at predetermined intervals.

In further embodiments, the fermenter system 100 is configured to convey a portion of the nutrient-rich source 140 to any fermenter, including the first 100a or last fermenter 110n, at any interval. Alternatively, the fermenter system is configured to convey a portion of the carbon-rich source 130 or biomass to any fermenter, including the first or last fermenter, at any interval. Further, the fermenter system is configured to convey any portion of the fermentation broth to any fermenter, including the first 100a or last fermenter 110n, at any interval. Further, the fermenter system 100 is configured to convey a portion of undigested biomass or partially digested biomass from any fermenter to any other fermenter at any interval via the conduits 120.

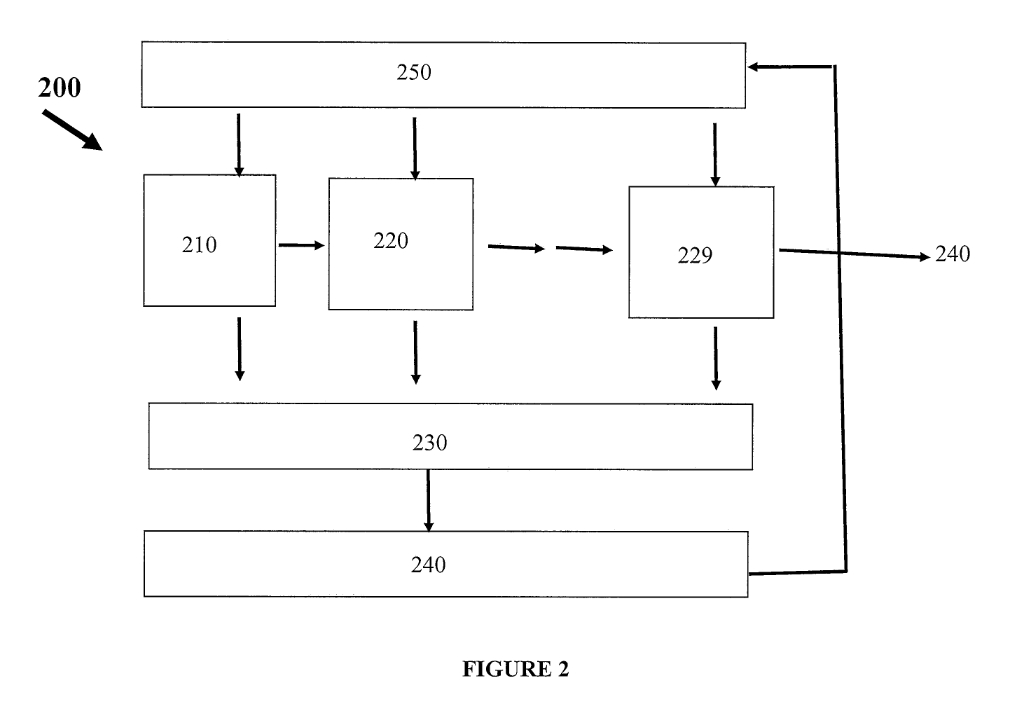

METHOD: Referring now to FIG. 2, the method 200 generally comprises a first fermentation step 210, a second fermentation step 220, measuring 230, and analyzing 240, and fermentation optimization 250. In embodiments, the first fermentation step 210 is initiation of biomass fermentation. In certain instances, the second fermentation step 220 may be any of a plurality of successive fermentation steps for the biomass, for example a third fermentation step, a fourth fermentation step, up to a last fermentation step 229. In embodiments, each of the fermentation steps is occurring at substantially similar time. In embodiments, the measuring 230 comprises measuring at least one property of each of the fermentation steps 210, 220, 229, etc. The measured properties are analyzed 240, for example compared to each other or compared to a predetermined property measurement. The analyzed measurements are then utilized to determine fermentation optimization 250 in the fermentation steps 210, 220, 229, etc to maximize production 260. Examples of fermentation optimization 250 may include introducing additional material, and nutrients, altering material and nutrient ratios, or altering the fermentation step order.

Referring to FIG. 1 and FIG. 2, in more detail, the method relates to the introduction of carbon sources 130, nutrient sources, 140 or combinations thereof to one or more of the fermenters 110 in the fermenter system 100 to increase the efficiency and yield of carboxylate products. The method comprises introducing additional nutrient sources to one or more fermenters. Alternatively, the method comprises the introduction of additional carbon sources 130 to at least one fermenter 110. In additional alternate embodiments, the method comprises the exchange of biomass, fermentation broth, or both between two or more fermenters 110.

More specifically, the method of the present disclosure relates to a plurality of fermentation stages that are producing concentrated fermentation broth by digestion of biomass. The properties of the fermentation broths are measured by sensors, compared between each of the fermentation stages, and additional carbon sources or nutrients are introduced to the fermentation stages based on the comparison. Alternatively, the fermentation broth properties are compared against a predetermined property. Also, the concentrated fermentation broths and partially digested biomasses may be exchanged such that a first digested biomass is introduced to a second fermentation broth, and vice versa. In embodiments the method optimizes the carbon-source, nutrient, and fermentation broth properties to increase carboxylate production.

In embodiments a property of any of the fermentation broths is detected and analyzed prior to introducing a nutrient, a carbon-source, or combinations thereof to the fermentation broth. In certain embodiments, detecting a property of the fermentation broth comprises measuring carboxylate product concentration, carbon concentration, nutrient concentration, suspended solid concentration, biomass to carboxylate conversion rates, and any other fermentation metabolite parameters. Further, analyzing comprises determining the differences in the property between more than one fermentation broths, for example from more than one fermentation steps. Alternatively, analyzing may comprise determining the differences in the property between at least one fermentation broth and a predetermined value. The differences may be used to determine additional carbon or nutrient introductions to the fermenter system.

In a non-limiting example, determining the soluble portion to insoluble portions change for a given nutrient concentration assists in determining the fermentation activity in a fermenter, because rapidly growing and dividing microorganisms take up soluble portions and convert them to proteins and other insoluble, intracellular macromolecules. As such the soluble and insoluble portions of the carbon and nutrient concentration in the fermentation may be measured by a sensor in the fermentation broth and analyzed by determining the differences between a first fermentation broth and a second fermentation broth. This analysis in turn determines the rate at which carboxylate products are being produced, whether a carbon or nitrogen source is required to maintain optimal fermentation conditions, whether the fermentation broth or biomass is ready for introduction to another fermenter, or alternatively, whether the fermentation broth is sufficiently concentrated to be withdrawn from the fermentation system for product isolation.

In an embodiment, the method comprises biomass-fermentation broth with staged nutrient injection. Prior to the first fermentation step, the biomass maybe partially digested prior to or it may be fresh, undigested biomass. The first fermentation step is inoculated and fermented to form a first fermentation broth and a first digested biomass. The properties of a nutrient in the first fermentation broth are detected by a sensor. The first digested biomass is introduced to a second fermenter having a second fermentation having a second fermentation broth for the second fermentation step. In certain instances, the nutrient properties of the second fermentation broth are detected by a sensor. The second fermentation step forms a second digested biomass and a third fermentation broth. The properties of the nutrient in the third fermentation broth are detected by a sensor. The properties of the nutrient in first, second, and third fermentation broths are compared as described previously. If the comparison or analysis shows that the nutrient property of any of the fermentation steps is outside a predetermined range or has depleted below a predetermined level, additional nutrients are introduced to the first fermentation step, the second fermentation step, or both. Alternatively, if the nutrient properties are within the predetermined range or above the predetermined threshold in one of the fermentation steps no additional nutrients are added to that fermentation step.

In another embodiment, the method comprises biomass-fermentation broth with staged carbon source injection. Prior to the first fermentation step, the biomass maybe partially digested prior to or it may be fresh, undigested biomass. The first fermentation step is inoculated and fermented to form a first fermentation broth and a first digested biomass. The properties of a carbon source in the first fermentation broth are detected by a sensor. The first digested biomass is introduced to a second fermenter having a second fermentation broth for the second fermentation step. In certain instances, the carbon source properties of the second fermentation broth are detected by a sensor. The second fermentation step forms a second digested biomass and a third fermentation broth. The properties of the carbon source in the third fermentation broth are detected by a sensor. The properties of the carbon source in first, second, and third fermentation broths are compared as described previously. If the comparison or analysis shows that the carbon source property of any of the fermentation steps is outside a predetermined range or has depleted below a predetermined level, additional carbon sources are introduced to the first fermentation step, the second fermentation step, or both. Alternatively, if the carbon source properties are within the predetermined range or above the predetermined threshold in one of the fermentation steps no additional nutrients are added to that fermentation step.

In another embodiment, the method comprises biomass-fermentation broth counter-current with staged nutrient and carbon source injection. Prior to the first fermentation step, the biomass maybe partially digested prior to or it may be fresh, undigested biomass. The first fermentation step is inoculated and fermented to form a first fermentation broth and a first digested biomass. The properties of the nutrient and carbon source in the first fermentation broth are detected by a sensor. The first digested biomass is introduced to a second fermenter having a second fermentation broth for the second fermentation step. In certain instances, the nutrient and carbon source properties of the second fermentation broth are detected by a sensor. The second fermentation step forms a second digested biomass and a third fermentation broth. The properties of the nutrient and carbon source in the third fermentation broth are detected by a sensor. The properties of the nutrient and carbon source in first, second, and third fermentation broths are compared as described previously. If the comparison or analysis shows that the nutrient and carbon source properties of any of the fermentation steps are outside a predetermined range or have depleted below a predetermined level, additional nutrients and carbon sources are introduced to the first fermentation step, the second fermentation step, or both. Alternatively, if the nutrient and carbon source properties are within the predetermined range or above the predetermined threshold in one of the fermentation steps no additional nutrients are added to that fermentation step. In certain instances, the ratio between the nutrient and carbon source concentration is the property that is detected. When the nutrient concentration or the carbon source concentration change, the ratio between them changes. If the ratio between the nutrient concentration and the carbon source concentration falls outside of a predetermined range or below a predetermined, either the nutrient or the carbon source will be introduced to the first fermentation step, the second fermentation step, or both. In further instances, it may be envisioned that the nutrient and the carbon source are each introduced to a different fermentation step.

In another embodiment, the method comprises biomass-fermentation broth with staged fermentation broth and biomass exchange. Prior to the first fermentation step, the biomass maybe partially digested prior to or it may be fresh, undigested biomass. The first fermentation step is inoculated and fermented to form a first fermentation broth and a first digested biomass. The properties of the first fermentation broth are detected by a sensor. The first digested biomass is introduced to a second fermenter having a second fermentation broth for the second fermentation step. In certain instances, the properties of the second fermentation broth are detected by a sensor. The second fermentation step forms a second digested biomass and a third fermentation broth. The properties of the third fermentation broth are detected by a sensor. The properties first, second, and third fermentation broths are compared as described previously. If the comparison or analysis shows that the properties of any one of the fermentation steps are outside a predetermined range or have depleted below a predetermined level in the first, second, or third fermentation broths but are still high in another one of the first, second, or third fermentation broths, the fermentation broths may be exchanged. In other words the high property fermentation broth may be exchanged for the depleted property fermentation broth in any one of the fermentation steps. Additionally, it may be envisioned that a portion of the fermentation broths may be exchanged.

In certain embodiments, the present disclosure relates to a method of automatically adjusting a plurality of fermentations by the addition of predetermined nutrients, carbon sources, or combinations thereof. In certain instances, a computer is connected to the each of the sensors associated with the fermenters. The computer records the sensor reading onto a computer readable medium. The computer further comprises an algorithm for comparing the sensor readings, determining which readings may be remedied by at least one of the steps previously disclosed herein, accessing instructions stored on the computer readable medium, and distributing the instructions automatically to the fermenters, conduits, nutrient-sources and carbon sources such that the fermentation broth, biomass, and carbon or nutrients are staged to maximize carboxylate production.

CARBON-NUTRIENT CONCENTRATION. As may be understood by a skilled artisan, the biomass in the last fermenter has reduced nutrient-rich components and fermentation of the remaining material produces a low yield fermentation broth. With each more recently inoculated stage, the biomass is less digested, allowing the fermentation broth to increase product concentrations without increasing the concentration of inhibitors. However, as the biomass is increasingly digested a reduction of nutrient sources, carbon sources, or a combination thereof inhibits microorganism growth and reduces the carboxylic acid yield. Particularly, in later biomass fermenter stages (i.e. 4^{th} fermenter, 5^{th} fermenter, etc) these nutrients are reduced, which in turn inhibits or reduces the effectiveness of the dilute fermentation broth. Further, with the dilute fermentation broth unable to efficiently ferment the digested biomass, the subsequent introductions to less digested biomass, the anaerobic population is reduced, the exponential growth phases does not reach peak population before the inhibition of the fermentation process, the efficiency of fermentation lower and lowering the final yield of the carboxylate product. In this embodiment, the addition of a nutrient

Additionally, as may be understood by a skilled artisan, typically the introduction of a nutrient-rich or a carbon-rich component to the first fermenter may not be favorable for the overall economics of the present disclosure. The fermentation broth in the first fermenter has the highest carboxylate product concentration and the highest biomass concentration. The addition of further nutrients is not going to push the carboxylate production beyond the point of inhibition and the nutrients are less likely to be consumed given the highest concentration of undigested biomass is also found in the first fermenter. As such, the nutrients would be withdrawn with the carboxylate product, separated, and either destroyed or recycled back into the fermenter system. Further, the high nutrient concentrations may interfere with separation and purification of carboxylate products for downstream processing. The addition of carbon-rich components into a fermenter having the highest concentration of biomass will not result in efficient degradation of the biomass, as the anaerobes preferably utilized the soluble and suspended nutrients first. However, in the instances of multiple pass or recycling fermentations, or nutrient and carbon-poor biomass, it may be beneficial to introduce nutrients or carbon-components to the first fermenter.

While the preferred embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

While the preferred embodiments of the invention have been shown and described, modifications thereof can be made by one skilled in the art without departing from the spirit and teachings of the invention. To further illustrate various illustrative embodiments of the present invention, the following examples are provided.

### EXAMPLES

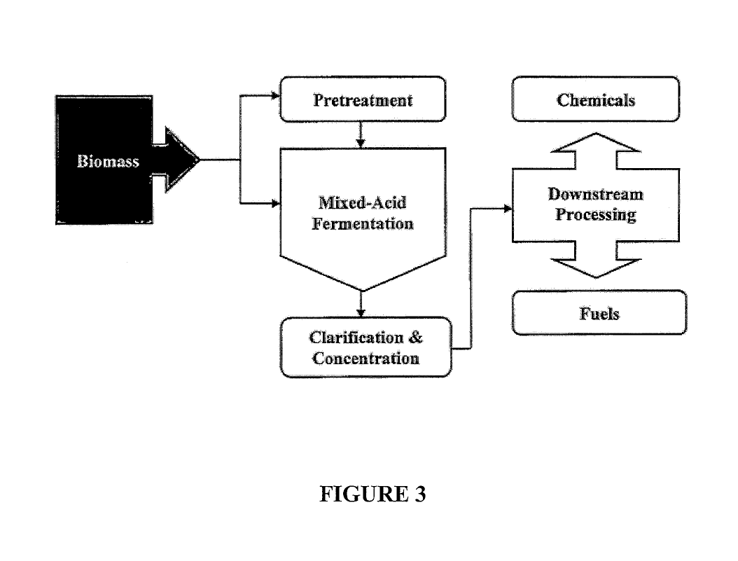

EXPERIMENTAL BACKGROUND Referring now to FIG. 3, illustrating a block flow diagram of the mixed-acid fermentation, carboxylate production system. Without limitation by theory, the step of fermenting biomass represents a rate-limiting step in the production of carboxylic acids. In instances, maintaining sufficient nutrient concentrations, including nitrogen, through the fermentation system influences and determines fermentation yield. Further, the MixAlco process functions optimally with high yield fermentation products, and more specifically, with high yield carboxylate production.

The fermentation yield is related to the carbon-nitrogen ratio and nitrogen within the fermentation broth. Without limitation, the fermentation broth comprises a mixture of insoluble and soluble bio-materials. Further, the fermentation broth comprises soluble and insoluble nitrogen, and the concentrations in these states do not alter the nitrogen flow within the fermenter. In certain instances, the soluble nitrogen concentration in the fermentation broth affects the carbon-nitrogen ratio more significantly than the insoluble nitrogen. As may be understood by one skilled in the art, the soluble nitrogen is accessible to the microorganisms in the fermenter.

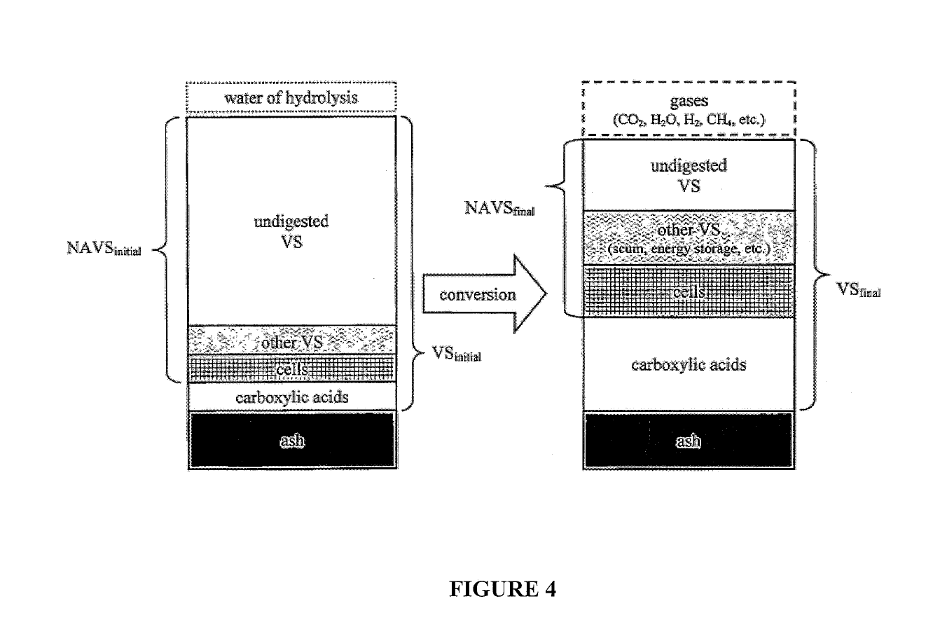

Referring now to FIG. 4, illustrating an approximate-proportion diagram of the conversion of biomass using mixed-acid fermentation. The feed consists of initial volatile solids (VSᵢₙᵢₜᵢₐₗ), composed of undigested volatile solids (VS) or other solids, including without limitation, scum, sludge, energy storage compounds, proteins, and other molecules. Further the VSᵢₙᵢₜᵢₐₗ comprises cells, and carboxylic acids. The carboxylic acids may be found in, for example, the nutrient source or other metabolites. The enzymes produced by the mixed-culture of acid-forming microorganisms hydrolyze polymers such as cellulose, and hemicellulose into simple sugars and monomers, which are subsequently fermented into carboxylic acids, and gases. In certain instances, the microorganisms form additional VS components that are found in the final volatile solid (VS_{final}) composition. Ash may be understood by an artisan to be inert and maintain at least approximately the same mass from fermenter entry to exit. The conversion of from VSᵢₙₜᵢₐₗ to VS_{final} may be considered as the conversion of VS digested per VS fed. Alternatively, the difference of VSᵢₙₜᵢₐₗ and VS_{final}, compared to VS_{final}.

In certain instances, the mixed-acid fermentations digest a wide variety of biological components, including cellulose, hemicelluloses, starch, free sugars, pectin, proteins, fats, and dead cells. Where the digested portions of these cellular components, and including cells, extracellular proteins, energy-storage compounds, and waste scum, are volatile solids, they must be considered products. Further, certain anaerobic cultures ferment lignin to some extent. As may be understood by one skilled in the art, all the VS, except for carboxylic acids, represent potential reactants. Hereinafter, these reactants are defined as non-acid volatile solids (NAVS). Without limitation by theory, this definition simplifies the complicated reaction system into four quantifiable and industrially meaningful terms: water, ash, acid, and NAVS.

As FIG. 4 illustrates the conversion of biomass in fermentation, the water of hydrolysis may be estimated by assuming the biomass is predominately cellulosic and has a monomer weight molecular weight of 162 g/mol. When a cellulose monomer is hydrolyzed, it gains one mole of water as found in Equation 1:

water of hydrolysis (g)=NAVS_{consumed}(g)18/162 (1)

Further, as disclosed hereinabove, a portion of the carboxylic acids in VS_{final} is found in the VSᵢₙᵢₜᵢₐₗ. In experimental instances, for example, where chicken manure is used as a nutrient source in VSᵢₙᵢₜᵢₐₗ, the feed contains a significant concentration of organic acids. As in the chicken manure example, ˜45 g/L(liq), contributes ˜0.022 g acid/g NAVS fed. Without limitation by theory, the failure to account for the carboxylic acids in the feed, the actual acid production of the fermentation system is unclear. For the purpose of this discussion, four definitions of yield (Equations 10-13) are introduced herein below with respect to different points in the fermentation system: feed, exit streams, microbial culture, and product stream.

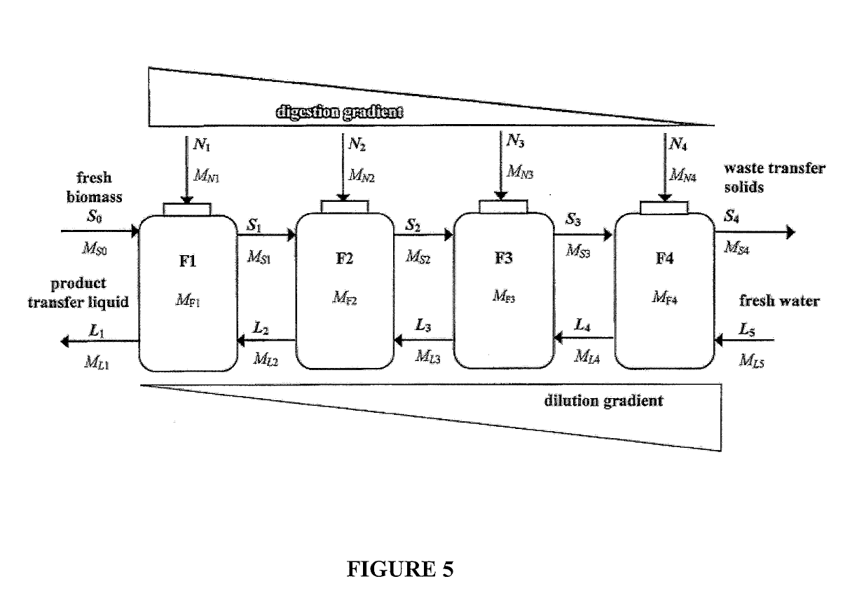

Referring now to FIG. 5 which illustrates a four-stage (F1-F4) countercurrent fermentation train with digestion and liquid dilution gradients determined by the introduction of fresh biomass (S₀) and product transfer liquid (L₀). The moisture contents of the nutrients (M_{N#}), fresh biomass (M_{S#}), and transfer liquid (M_{L#}) are each determined for each stage (F1-F4). Without limitation by any particular theory, this disclosure may be extrapolated to a fermentation train having n fermenter stages, wherein n is any interger. Additionally, the following nitrogen contents and ratio abbreviations and reference numerals will be used herein:

v≡nitrogen content (g N/g wet biomass), and

η≡soluble nitrogen trials fraction (g soluble N/g total N)

M_{Xi}=the moisture content (g moisture/g wet sample) of Stream or Material Xi

In order to optimize the acid production of a fermenter train in this configuration to a predictable concentration, there are methods for determining nutrient feed. Further, this method or model alters the masses of the nutrients (N#) fed to each fermenter (F1-F4(Fₙ)). First, wherein the biomass (S_{#}) and liquid (L_{#}) flowrates are unknown, for instance in a new or altered operation fermentation train, using the mass balances within the system to determine flowrates which are then used to determine the optimal amount of nutrient to be fed to each fermenter. And alternatively, when the biomass (S_{#}) and liquid (L_{#}) flowrates are known, the actual flowrates are used to the optimal amount of nutrient to be fed to each fermentor (F1-F4(Fₙ)).

For the biomass (S_{#}) and liquid (L_{#}) flowrates determination through the fermenter train, the nitrogen mass in the calculation between total, solid, and moisture mass balances is negligible to the total mass. As such, using two of the three masses it is possible to determine the biomass (S₀) and liquid (L₀) flowrates. The calculation under steady-state operation at any particular fermenter, and in this exemplary instance calculated for and arbitrary ith, is determined by the following equations.

For the total mass balance:

$$\frac{d ( {F}_{i} )}{d t} = 0 = {S}_{i - 1} + {L}_{i + 1} + {N}_{i} - {S}_{i} - {L}_{i} \qquad ( 2 )$$

For the total moisture balance:

$$\begin{matrix}\frac{d ( {F}_{i} {M}_{{F}_{i}} )}{d t} = 0 \\ = {S}_{i - 1} {M}_{{S}_{i - 1}} + {L}_{i + 1} {M}_{{L}_{i + 1}} + {N}_{i} {M}_{{N}_{i}} - {S}_{i} {M}_{{S}_{i}} - {L}_{i} {M}_{{L}_{i}}\end{matrix} \qquad ( 3 )$$

For the total dry solids balance:

$$\begin{matrix}\frac{d ( {F}_{i} ( 1 - {M}_{{F}_{i}} ) )}{d t} = 0 \\ = {S}_{i - 1} ( 1 - {M}_{{S}_{i - 1}} ) + {L}_{i + 1} ( 1 - {M}_{{L}_{i + 1}} ) + \\ {N}_{i} ( 1 - {M}_{{N}_{i}} ) - {S}_{i} ( 1 - {M}_{{S}_{i}} ) - {L}_{i} ( 1 - {M}_{{L}_{i}} )\end{matrix} \qquad ( 4 )$$

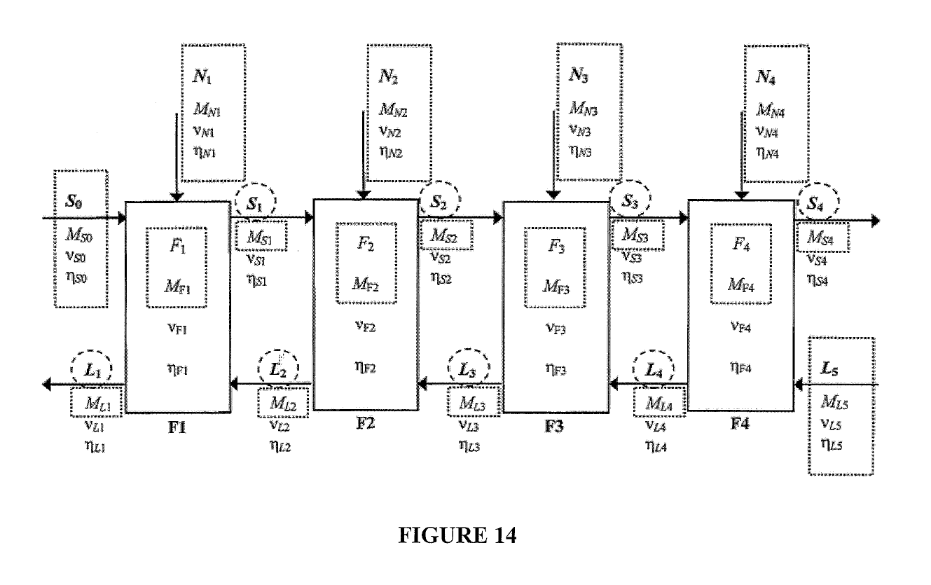

As such the biomass (S₀) and liquid (L₀) flowrates through the fermenter train may be calculated. Further, wherein the system of equations found in FIG. 14 use the total mass and the total moisture mass balances to determine the biomass (S_{#}) and liquid (L_{#}) flowrates through the fermentation train. In instances, calculation of the nitrogen balances is unnecessary, as the total mass balance accounts for nitrogen in both total dry solids, and total moisture. In certain instances, the mass balances, may be then used in the second model to optimize the nitrogen balance between soluble and insoluble mass to further optimize the acid production.

Determining the mass balance between soluble and insoluble nitrogen factors the flowrates of the biomass (S_{#}) and liquid (L_{#}) into the mass balance. This balance is calculated by the following equations:

For soluble nitrogen mass balance:

$$\begin{matrix}\frac{d ( {F}_{i} {v}_{{F}_{i}} {η}_{{F}_{i}} )}{d t} = 0 \\ = {S}_{i - 1} {v}_{{S}_{i - 1}} {η}_{{S}_{i - 1}} + {L}_{i + 1} {v}_{{L}_{i + 1}} {η}_{{L}_{i + 1}} + {N}_{i} {v}_{{N}_{i}} {η}_{{N}_{i}} - \\ {S}_{i} {v}_{{S}_{i}} {η}_{{S}_{i}} - {L}_{i} {v}_{{L}_{i}} {η}_{{L}_{i}}\end{matrix} \qquad ( 5 )$$

For insoluble nitrogen mass balance:

$$\begin{matrix}\frac{d ( {F}_{i} {v}_{{F}_{i}} ( 1 - {η}_{{F}_{i}} ) )}{d t} = 0 \\ = {S}_{i - 1} {v}_{{S}_{i - 1}} ( 1 - {η}_{{S}_{i - 1}} ) + {L}_{i + 1} {v}_{{L}_{i + 1}} \\ ( 1 - {η}_{{L}_{i + 1}} ) + {N}_{i} {v}_{{N}_{i}} ( 1 - {η}_{{N}_{i}} ) - \\ {S}_{i} {v}_{{S}_{i}} ( 1 - {η}_{{S}_{i}} ) - {L}_{i} {v}_{{L}_{i}} ( 1 - {η}_{{L}_{i}} )\end{matrix} \qquad ( 6 )$$

However, it should be noted that this calculation accepts the fermenter operating assumptions including ideal mixing in each stage; within a stage, the liquid-phase nitrogen concentration is uniform, such that the concentration of nitrogen in the free liquid and liquid absorbed in the transfer solids are identical; and the solid-phase nitrogen concentration is uniform at least within biomass (S₀), liquid (L₀) and bulk (Fᵢ) transfer streams. The relationship of these streams is further determined by the equations:

For soluble nitrogen mass:

$$\frac{{v}_{{S}_{i}} {η}_{{S}_{i}}}{{M}_{{S}_{i}}} = \frac{{v}_{{L}_{i}} {η}_{{L}_{i}}}{{M}_{{L}_{i}}} = \frac{{v}_{{F}_{i}} {η}_{{F}_{i}}}{{M}_{{F}_{i}}} = \frac{g soluble nitrogen}{g liquid} \qquad ( 7 )$$

And, insoluble nitrogen mass:

$$\frac{{v}_{{S}_{i}} ( 1 - {η}_{{S}_{i}} )}{( 1 - {M}_{{S}_{i}} )} = \frac{{v}_{{L}_{i}} ( 1 - {η}_{{L}_{i}} )}{( 1 - {M}_{{L}_{i}} )} = \frac{{v}_{{F}_{i}} ( 1 - {η}_{{F}_{i}} )}{( 1 - {M}_{{F}_{i}} )} = \frac{g in soluble nitrogen}{g dry solid} \qquad ( 8 )$$

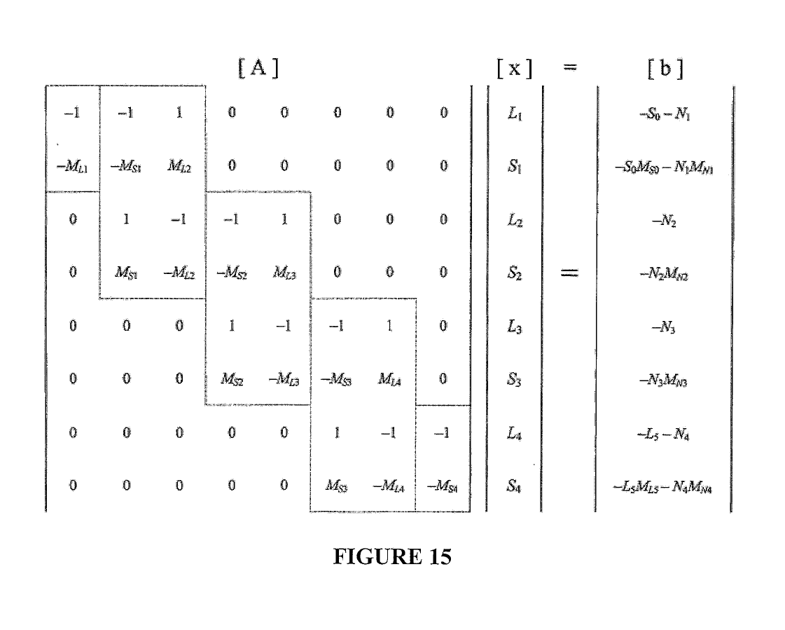

The unknown terms in Equations (5)-(8), are further solved for according to the equations of FIG. 15. And once the nitrogen properties for the streams has been determined, further calculating the nitrogen properties of the biomass at each stage (F1-F4) may be done by Equations (7)-(8).

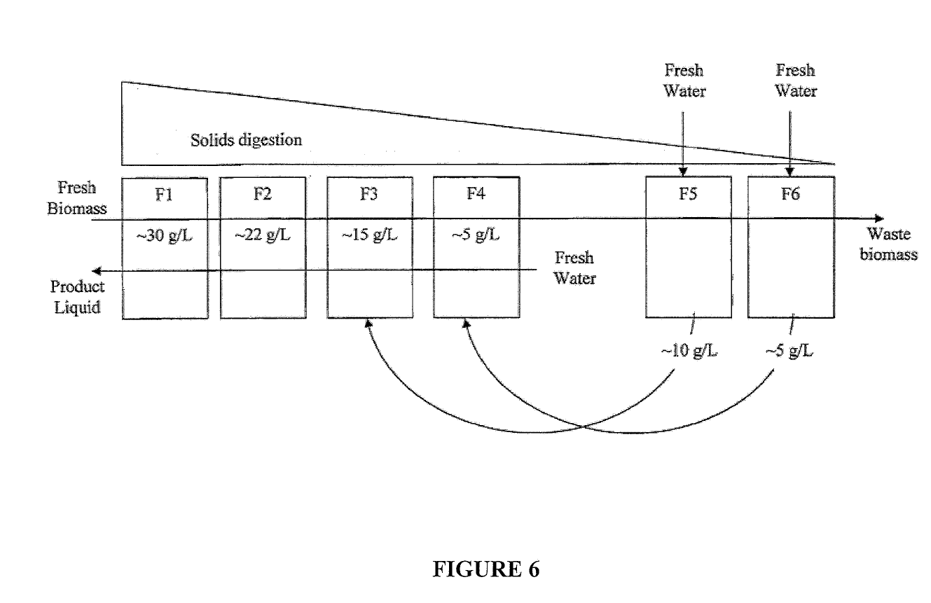

Referring now to FIG. 6, illustrating an alternate fermenter train configuration. It is possible that the mass balance Equations (2)-(8) herein are applied in a cross-flow train configuration, wherein the mass balance of biomass (S_{#}), liquid (L_{#}) and bulk (Fᵢ) transfer streams has a reduced product. Without limitation by theory, by reducing the products in later stages, as in the exemplary illustration F5-F6, would reduce inhibition. The reduction of by removing a portion liquid to a fermenter with an acid concentration approximately the same as, or nearest to that from which the liquid was removed. As may be understood by one skilled in the art, the numbers of the fermenter stages are exemplary only, and there may be more or less fermenter stages.

### Example 1

The MixAlco process is a “biorefinery” that converts any biodegradable biomass into useful chemicals and fuel. Although some substrates (e.g., food scraps and office paper) are easily digested, most lignocellulosic biomass must be pretreated with lime and oxygen/air to increase digestibility. The biomass is then fermented by a mixed culture of acidogens to produce two- to seven-carbon carboxylic acids, which are buffered with calcium carbonate or ammonium bicarbonate. The fermentation broth is clarified, concentrated, and dried to produce carboxylate salts, a “biocrude” that can be chemically converted to chemicals and fuels.

Acid fermentation is a key step in the MixAlco process because it dominates the capital costs, and determines the overall rates and yields. Mixed-culture acid fermentation is ideal for a biorefinery for the following reasons: no enzyme addition, no genetically modified microorganisms or mono-cultures, no contaminates, adapts to feedstock fluctuations, and low capital and operating costs. The mixed-culture acid fermentation employs similar microorganisms as biomethane fermentations, except methanogens are inhibited with iodoform.

Typically, two to four fermenters are used to create a countercurrent fermentation “train”. The first fermenter is fed with the most reactive (fresh) biomass, but has the highest product carboxylic acid concentration (greatest product inhibition). The last fermenter has the most recalcitrant (digested) biomass, but has the lowest product concentration (least product inhibition). This countercurrent strategy achieves both high product concentration and high conversion. Carbohydrates (e.g., municipal solid waste, paper, sugarcane bagasse) and nutrients (e.g., sewage sludge, manure) ferment better when blended in an optimal ratio. In past disclosures, nutrients have been treated as though they were insoluble dry solids and were fed to fermenter (F₁) along with the insoluble carbohydrates (S₀). This practice was understandable because nutrients were typically dried for convenient laboratory use. Recently, it was determined many nutrients are soluble and can leave with the product transfer liquid (L₁), as in FIG. 5, before being incorporated into microbial cells and enzymes. Further, carbon-nitrogen ratios (C/N) were not measured or controlled in these fermentations. Thus, it is probable that performance was restricted by nitrogen and nutrient limitations, rather than the feedstock or operating conditions.

Carbon-nitrogen ratio (C/N) Mixed-culture acid fermentations of lignocellulose are long (20-60 days liquid retention) and dilute (20-40 g acid/L), thus requiring large fermenters. Improving fermentation performance will significantly reduce capital costs and increase productivity. Nitrogen is required for cell replication, maintenance, metabolism, and production of enzymes. Because lignocellulose hydrolysis is the rate limiting step, maintaining sufficient nitrogen concentrations/proportions is necessary to ensure that production of critical hydrolysis enzymes like cellulase is not restricted. In biomethane fermentations, the carbon-to-nitrogen (C/N) ratio influences performance. Too much nitrogen may result in ammonium toxicity and too little nitrogen limits cellular activity; therefore, nitrogen control is necessary for optimum performance. For countercurrent mixed-acid fermentations, no models currently exist that describe nitrogen behavior. In this disclosure, the carbon-nitrogen ratio is defined as the mass of total organic carbon minus the carbon contributed by the carboxylic acids (product) (g non-acid carbon; g CNA) per mass of nitrogen (g N). With respect to acidogens, this definition of C/N ratio characterizes the relative proportion of reactant (energy) per nitrogen (nutrient). The organic acids represented 1-8% of the total carbon. If the carbon contributed by the acids is not excluded, the C/N will be overstated, which could lead to over-addition of nutrients (added cost) and sub-optimal performance as above.

For similar fermentations (methane and hydrogen), a wide range of optimal C/N (10-90 g/g) and where 30 is the most cited optimum for producing carboxylic acids disclosed. Because the C/N ratio is reported in a variety of units and there are conflicting scopes of research, the present disclosure determines methods for finding the optimum C/N ratio and the nitrogen mass balance for mixed-acid fermentations and operating the fermenter train accordingly. This Example assumes 30 g CNA/gN is the optimal C/N ratio.

Methods Table 1 lists the feedstock properties. Shredded office paper (carbohydrate source) from Texas A&M University's recycling center (College Station, Tex.) and fresh (wet)

TABLE 1

| | Office Paper | Fresh Chicken Manure |
|---|---|---|
| Moisture content, M | 0.051 ± 0.03 | 0.660 ± 0.03 |
| (g H₂O/g wet sample) | | |
| Ash content, I | 0.130 ± 0.06 | 0.592 ± 0.09 |
| (g ash/g dry sample) | | |
| Carbon content, C | 36.3 ± 0.8 | 8.35 ± 0.7 |
| (g C/g wet sample) | | |
| Nitrogen content, N | 0.25 ± 0.07 | 1.10 ± 0.2 |
| (g N/g wet sample) | | |
| Carbon-nitrogen ratio | 138.3 ± 43 | 7.73 ± 0.7 |
| (g C_{NA}/g N) | | |
| Soluble nitrogen fraction, η | ~0 | 0.419 ± 0.04 |

Error values represent one standard deviation

chicken manure (nutrient source) from Feathercrest Farm (Bryan, Tex.) were used in a 4:1 carbohydrate to nutrient ratio on a dry mass basis. Paper was selected because it is free of lignin and did not require pretreatment. No additional nutrients (bloodmeal, urea, etc.) were added, and as such the C/N ratio of the feed was 39.1 g CNA/g N.

Deoxygenated water was prepared by boiling de-ionized water to liberate dissolved oxygen gas. After cooling to room temperature in a covered vessel, 0.275 g sodium sulfide and 0.275 g cysteine (reducing agents) were added per liter of water. A small amount (80 pL) of methanogen inhibitor (20 g iodoform/L200-proof ethanol) was added to each fermenter bottle. The inoculum was obtained from the MixAlco Pilot Plant (College Station, Tex.), which was originally inoculated with marine microorganisms from Galveston, Tex. The mixed cultures were dominated by Clostridia species.

Analysis Ultra-centrifuged (15,000 rpm) fermentation liquid was mixed with equal parts of internal standard (1.162 g/L 4-methyl-n-valeric acid) and 3-M H₃PO₄. The H₃PO₄ ensures that carboxylate salts are converted to carboxylic acid prior to analysis. The carboxylic acid concentration was measured using an Agilent 6890 Series Gas Chromatograph (GC) system equipped with a flame ionization detector (FID) and an Agilent 7683 automatic liquid sampler. A 30-m fused-silica capillary column (J&W Scientific Model #123-3232) was used. The column head pressure was maintained at 2 atm (absolute). After each sample injection, the GC temperature program raised the temperature from 40° C. to 200° C. at 20° C./min. The temperature was subsequently held at 200° C. for 2 min, with a total run time per sample of 11 min. Helium was the carrier gas. The calibration standard was volatile acid mix (Matreya, LLC, Cat. No. 1075).

Each fermenter was vented daily to relieve pressure and prevent rupture. The gas volume was measured by liquid displacement using an inverted graduated glass cylinder filled with an aqueous solution of 300 g CaCl₂/L to prevent microbial growth and carbon dioxide absorption. To monitor methane, 5 mL gas samples were taken through the fermenter septum, gas samples were analyzed by the Agilent 6890 Series Chromatograph with a thermal conductivity detector (TCD). Samples were injected manually. A 4.6 m stainless steel packed column with 2.1 mm ID (60180 Carboxen 100, Supelco 1-2390) was used. The inlet temperature was 230° C., the detector temperature was 200° C., and the oven temperature was 200° C. The total run time was 10 min. Helium was the carrier gas.

The C/N ratio was characterized using total carbon and total nitrogen contents, both of which were measured in a single test using an Elementor Variomax CN. Total organic carbon is preferred in the C/N ratio, but because 99% of the total carbon fed was organic carbon, the added cost of distinguishing the two was not justified. The C/N ratio was used to compare trends among the different nutrient feeding strategies. Because each train was fed the same feedstocks, these trends are similar, regardless of whether total carbon or total organic carbon was used. No external buffer, such as calcium carbonate, was added because minerals in the feed self-regulated the pH between 5.5 and 6.5. Total carbon and total nitrogen contents (g1100 g) were determined by Texas A&M University Soil, Water, and Forage Testing Lab (College Station, Tex.).

Moisture contents (M_{Xi}) and ash contents (I_{Xi}) were measured in series. First the sample was dried in a 105° C. forced-convection oven (>12 h) and then ashed in a 550° C. furnace (>3 h). Before drying, 3 g Ca(OH)₂/100 g sample was added to ensure all volatile acids were converted to salts and retained during drying. This practice disproportionately overstates the ash content; thus, exit-stream ash data were unreliable. To overcome this problem, the consumption of nonacid volatile solids (NAVS) was determined using the inert-ash approach.

Further, referring to the labels in the FIG. 4, the following terms are additionally applicable:

$$N A V {S}_{feed} ( g ) ≡ sum of N A V S in {S}_{0} , {N}_{1} , {N}_{2} , {N}_{3} , {N}_{4} , and {L}_{5}$$

$$N A V {S}_{exit} ( g ) ≡ sum of N A V S in {S}_{4} and {L}_{1}$$

$$N A V {S}_{consumed} ( g ) ≡ N A V {S}_{feed} - N A V {S}_{exit}$$

$${A}_{feed} ( g ) ≡ sum of carboxylic acid in {S}_{0} , {N}_{1} , {N}_{2} , {N}_{3} , {N}_{4} , and {L}_{5}$$

$${A}_{exit} ( g ) ≡ sum of carboxylic acid in {S}_{4} , {L}_{1} , and any liquid samples removed from F 2 - F 4$$

$${A}_{produced} ( g ) ≡ {A}_{exit} - {A}_{feed}$$

$${A}_{{L}_{1}} ( g ) ≡ total carboxylic acid in {L}_{1}$$

$$conversion ≡ C ≡ \frac{N A V {S}_{consumed}}{N A V {S}_{feed}}$$

$${yield}_{feed} ≡ {Y}_{F} ≡ \frac{{A}_{feed}}{N A V {S}_{feed}}$$

$${yield}_{exit} ≡ {Y}_{E} ≡ \frac{{A}_{exit}}{N A V {S}_{feed}} = {Y}_{F} + {Y}_{C}$$

$${yield}_{culture} ≡ {Y}_{C} ≡ {Y}_{E} - {Y}_{F} ≡ \frac{{A}_{produced}}{N A V {S}_{feed}} = C \cdot E$$

$${yield}_{process} ≡ {Y}_{P} ≡ \frac{{A}_{{L}_{1}}}{N A V {S}_{feed}}$$

$$total acid selectivity ≡ E ≡ \frac{{Y}_{C}}{C}$$

$$total acid productivity ( train ) ≡ P ≡ \frac{{A}_{produced}}{TLV \times time}$$

Acetic acid equivalents (aceq) equate the reducing potential of a carboxylic acid mixture to an energy-equivalent mass of acetic acid. Concentrations are converted to acetic acid equivalents using the following equation:

$$α ( mol \text{/} L ) = acetic ( mol \text{/} L ) + 1.75 \cdot propionic ( mol \text{/} L ) + 2.50 \cdot butyric ( mol \text{/} L ) + 3.25 \cdot valeric ( mol \text{/} L ) + 4.0 \cdot caprioc ( mol \text{/} L ) + 4.75 \cdot heptanoic ( mol \text{/} L )$$

And on a mass basis the aceq are defined as:

$$aceq ( \frac{g}{L} ) = 60.05 ( \frac{g}{mol} ) \cdot α ( \frac{mol}{L} )$$

Measuring Performance During the steady-state period, the flowrate (amount/day) of acid, ash, NAVS, water, and gas were determined. The fermentations trains were semi-continuous with material transfers performed three times per week. To determine the flowrate of a component, the moving cumulative sum of that component was plotted with time. The component flowrate (amount/day) was determined from the slope of the line. All performance variables were calculated from component flowrates determined by the slope method.

The NAVS_{consumed} is the difference between the NAVS in the inlet and exit streams. This quantity can be determined by two approaches: direct measurement and inert ash. Direct measurement, uses the NAVS component flowrate in inlet and outlet streams (S₀, L_{S}, N₁, N₂, N₃, N₄, S₄, L₁) are measured directly using the slope method and the following equation:

$$N A V {S}_{{X}_{i}} = {X}_{i} ( ( 1 - {M}_{{X}_{i}} ) ( 1 - {I}_{{X}_{i}} ) - \frac{{[ A ]}_{{X}_{i}} {M}_{{X}_{i}}}{{ρ}_{W}} ( \frac{1 L}{1000 mL} ) )$$

where

Xᵢ=total transferred mass of Stream Xᵢ (g)

M_{X}_{ᵢ}=moisture content of Stream Xᵢ (g moisture/g wet sample)

I_{X}_{ᵢ}=ash content of Stream Xᵢ (g ash/g dry sample)

[A]_{X}_{ᵢ}=total carboxylic acid concentration (g/L_{Liq}) of Stream Xᵢ

ρ_{w}=density of water (1 g/mL)

The total inlet NAVS feed flowrate minus the NAVS exit flowrate equals the NAVS_{consumed} rate,

Assuming ash is inert, the ash flowrates in and out are equal. Then, based on this assumption, the difference between the dry material in the inlet and outlet streams results from the change in VS, not a change in ash. The NAVS_{consumed} rate (g NAVS_{consumed}/d) may be determined by the Equation:

$$\begin{matrix}N A V {S}_{consumed} rate = N A V {S}_{feed} rate - N A V {S}_{exit} rate \\ = ( Σ dry {solids}_{in} - Σ {ash}_{1 in} - Σ {acid}_{in} ) - \\ ( Σ dry {solids}_{out} - Σ {ash}_{in} - Σ {acid}_{out} ) \\ = ( Σ dry {solids}_{in} - Σ {acid}_{in} ) - \\ ( Σ dry {solids}_{out} - Σ {acid}_{out} )\end{matrix}$$

where:

dry solids in stream Xᵢ (g)=Xᵢ(1−M_{X}_{ᵢ})

$$acid in stream {X}_{i} ( g ) = {A}_{{X}_{i}} = \frac{{{X}_{i} [ A ]}_{{X}_{i}} {M}_{{X}_{i}}}{{ρ}_{W}} ( \frac{1 L}{1000 mL} )$$

The inert-ash approach was used to calculate conversion because it is independent of ash content measurements (which were inaccurate for this experiment). Ideally, both methods would give the same result.

Operation Liquid retention time (LRT) quantifies the average time for liquid to travel through the system. LRT influences the product concentration, and longer residence times allow for higher product concentrations:

$$LRT = \frac{TLV}{Q}$$

And where Q is determined using the slope method and Equation

$$\begin{matrix}Q = ( {L}_{5} {M}_{{L}_{5}} + {S}_{0} {M}_{{S}_{0}} + {\sum }_{i} {N}_{i} {M}_{{N}_{i}} ) \frac{1}{{ρ}_{W}} ( \frac{1 L}{1000 mL} ) \\ = total inlet liquid flowrate ( L \text{/} d ) \text{:}\end{matrix}$$

And TLV is the total liquid volume expressed as

$$T L V = {\sum }_{i} ( \frac{{K}_{Fi} {M}_{Fi}}{{ρ}_{W}} ( \frac{1 L}{1000 mL} ) + {L}_{Fi} )$$

where,

L₅, S₀, and Nᵢ are rates determined by the slope method (g/d)

K_{Fi}=the average mass of wet solid cake in Fermentor i (g),

L_{Fi}=the average volume of free liquid in Fermentor i (L).

Volatile solids loading rate (VSLR) quantifies the reactant feed rate relative to the total liquid volume and is defined as:

$$V S L R = \frac{N A V {S}_{feed} rate}{T L V}$$

VSLR is inversely related to conversion and yield. As VSLR increases, NAVS have less time to digest, which lowers conversion and yield. The NAVS concentration (SC_{Fi}) is defined as the ratio of reactant in Fi (NAVS_{Fi}) to the liquid volume in Fermenter Fi (LV_{Fi}):

SC_{Fi}≡NAVS_{Fi}/LV_{Fi}

where the acid concentration is directly proportional to SCTransfer solids physically appear solid, but have moisture contents of 0.70-0.85 g moisture/g total with all moisture fully absorbed in the biomass. Transfer liquids physically appear fluid, but may have 1-3% suspended solids. For a countercurrent staged fermentation (FIG. 5), there are six degrees of freedom. The following four operating parameters are completely independent: temperature, transfer frequency (transfer/time), solids retained in each fermenter (total mass), and liquid retained in each fermenter (total mass or volume). The remaining two operating parameters are selected from the following: reactant feed rate (S₀), waste transfer solid rate (S₄) (amount/transfer), liquid feed rate (L₅), and product transfer liquid rate (L₁) (amount/transfer). For laboratory fermentations, the reactant feed rate (S₀) and liquid feed rate (L_{S}) are typically held constant. For logistical reasons, large-scale operations may have to control the reactant feed rate (S₀) and product transfer liquid rate (L₁).

Table 3 summarizes the operating parameters of the five trains described herein. The normalized operating parameters (NOP) are calculated from the controllable operating moisture and ash contents, which are dictated by fermentation performance. Before a transfer, each fermenter and its contents were centrifuged at 4000 rpm. The liquid layer was decanted into a graduated cylinder and measured. The bottle with the remaining solid cake was weighed (Bᵢ), where “i” equals the fermenter number. For F1, the amount of transfer solids fed (S₀) was constant. For subsequent fermenters (Fi), the transfer solids fed was equal to the transfer solids removed (Sᵢ₋₁) from the previous fermenter plus the nutrient fed to that fermenter (Nᵢ). The transfer solids retained in each fermenter were controlled by a solids-retained-plus-bottle-weight set point (Wᵢ). The mass of transfer solids removed (Sᵢ) was determined by a simple material balance (Sᵢ=Bᵢ+Sᵢ₋₁+Nᵢ−Wᵢ). For each train, the solids-retained-plus-bottle-weight set point for F1 was 200 g and 300 g for F2 to F4. The set point for F1 was lower because fresh paper absorbed free transfer liquid added to F1. All decanted transfer liquid was transferred to the previous fermenter, as shown in FIG. 5.

To compare steady-state acid data, the two-tailed heteroscedastic student t-test (“TTEST” function in Microsoft Excel 2007) with a confidence level of 5% was used to calculate p-values. Unless otherwise stated, error bars represent a 95% confidence interval (two standard deviations). Sum-of-squared-errors techniques were used to determine the error of calculated values.

### Example 1

### Results

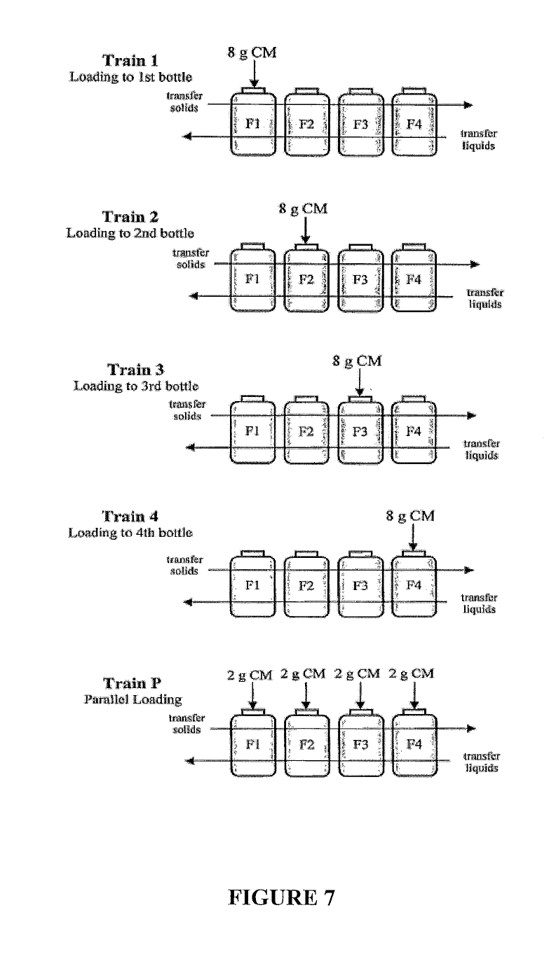

The four-bottle trains (FIG. 7) were run with identical operating parameters (Table 3), each with a different nutrient contacting pattern. Many variables influence fermentation performance (SC, VSLR, LRT, substrates, solid-liquid separation efficiency, number of stages, etc.). The interaction of operating parameters and nutrient addition strategies is not fully understood, so these results must be carefully interpreted and applied in context with operating parameters used in this study.

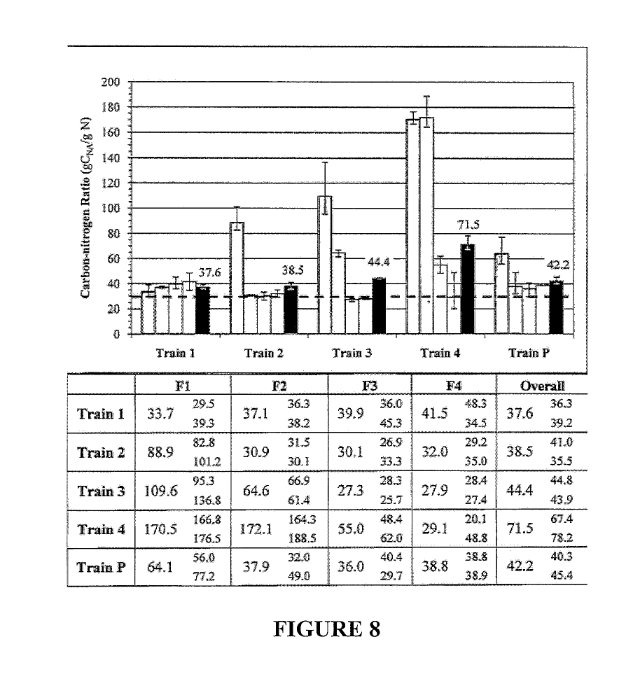

FIG. 8 shows the C/N ratio profile produced by each nutrient loading pattern. Overall C/N ratio is defined as the sum of non-acid carbon (g C_{NA}) in all fermenters divided by the sum of total nitrogen (g N) in all fermenters. Train 1 produced the most even C/N profile with ratios slightly increasing in successive stages. Train 2 had a high C/N ratio (90 g C_{NA}/g N) in F1, but F2-F4 had C/N ratios very close to the optimum of 30 g C_{NA}/gN. Train 4 had the most uneven C/N profile. Trains 3, 4, and P had overall C/N ratios greater than the feed (39±1 g C_{NA}/g N), indicating distribution inefficiencies and/or gaseous nitrogen loss. Each train had one or more bottles with a C/N ratio above 30 g C_{NA}/gN indicating nitrogen limitations; thus, no train was fully optimized.

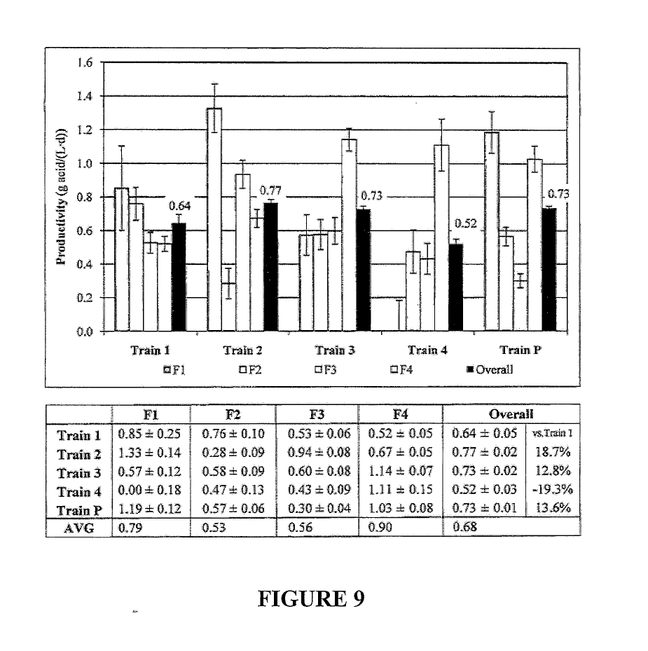

Total acid productivity is defined as the acid produced per liquid volume per day; thus, the acid contributed by the nutrient (chicken manure) is not included. FIG. 9 shows the productivity profile of each train. Overall productivities are weighted averages with the total liquid volume of each bottle. Although Trains 1 and 2 have virtually identical overall C/N ratios (37.6 and 38.5 g C_{NA}/g N, respectively), Train 2 had a much higher overall productivity (0.77 vs. 0.64 g acid produced/(L_{Liq.}·d)). This resulted because Train 2 had a greater percentage of its fermentation mass near the optimum C/N ratio than Train 1. In contrast, Train P had a higher C/N profile (42.2 g C_{NA}/g N, overall) and a higher productivity (0.73 g acid produced/(L_{liq.}·d)) than Train 1. This indicates the importance of non-nitrogen nutritional factors (e.g., phosphorus, minerals, etc.) and/or “freshness” of nutrients. F1 and F2 of Train 4 had similar C/N ratios around 170 g C_{NA}/g N, and similar steady-state acid concentrations around 13.8 g acid/L_{liq}. Despite receiving fresh paper, F1 of Train 4 had a productivity of zero, which indicates severe nitrogen and non-nitrogen nutrient limitations.

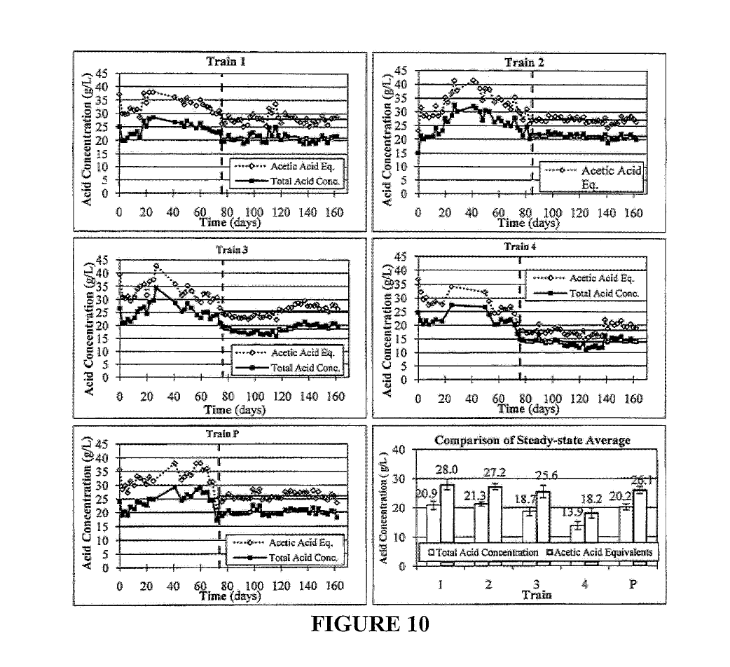

When comparing individual fermenters from each train, those that received the full amount of fresh nutrients did not have the highest productivity. A possible explanation for these phenomena is the carboxylic acid content (not the nutrients) of the chicken manure caused product inhibition that reduced productivity. FIG. 10 shows that total acid productivity depends on C/N ratio and increases as the C/N ratio approaches the optimum. The slope of the linear trend line indicates how sensitive a fermenter is to C/N ratio. F2 had the flattest slope indicating it was the least sensitive, whereas F4 had the steepest slope indicating the greatest sensitivity. This trend is understandable considering F4 contains the most recalcitrant biomass; thus, nutrients are critical for digestion. Further improvements in performance can be realized if optimal C/N ratios can be maintained in each fermenter. Using FIG. 10 to predict the productivity of each fermenter at a C/N of 30 g C_{NA}/g N suggests that overall productivities ranging from 0.83 to 0.99 g acid/(L·d) could be obtained (VSLR=7 g NAVS/(L_{liq.}·d) and LRT=15 d). If obtained, these productivities translate into culture yield improvements of 67-99% (0.13 8-0.165 g acid produced/g NAYS) verses Train 1.

Acid Concentration. Initially, the operating parameters did not produce transfer liquid from F1 because the paper loading rate (S₀) was too high relative to the water throughput (L₅); there was no free liquid because all liquid was absorbed in the fresh paper. To correct this, the solids-retained-plus-bottle-weight set point for F1 (W₁) of each train was decreased from 300 to 200 g (Day 20) and the water fed per transfer was increased from 175 to 300 mL per (Day 27). Thus, the noise/peak prior to steady state resulted from very high initial solids concentrations. Train 2 had the highest average steady-state acid concentration (21.3 g/L) with Trains 1, 3, 4, and P having concentrations of 20.9, 18.7, 13.9, and 20.2 g/L, respectively. The t-test showed that Train 2 was not significantly different than Train 1 (p=0.162). Train 1 had the highest average steady-state aceq concentration (28.0 g/L) with Trains 2, 3, 4, and P having concentrations of 27.2, 25.6, 18.2, and 26.1 g/L, respectively. Trains 1, 2, 3, and P had similar total acid and aceq product concentrations indicating that the nutrient loading pattern did not significantly affect product concentration. The ratio of aceq concentration to total acid concentration for Trains 1, 2, 3, 4, and P is 1.33, 1.28, 1.37, 1.31, and 1.29, respectively. Train 3 has a higher ratio than the other four trains indicating it produced more high-molecular-weight acids.

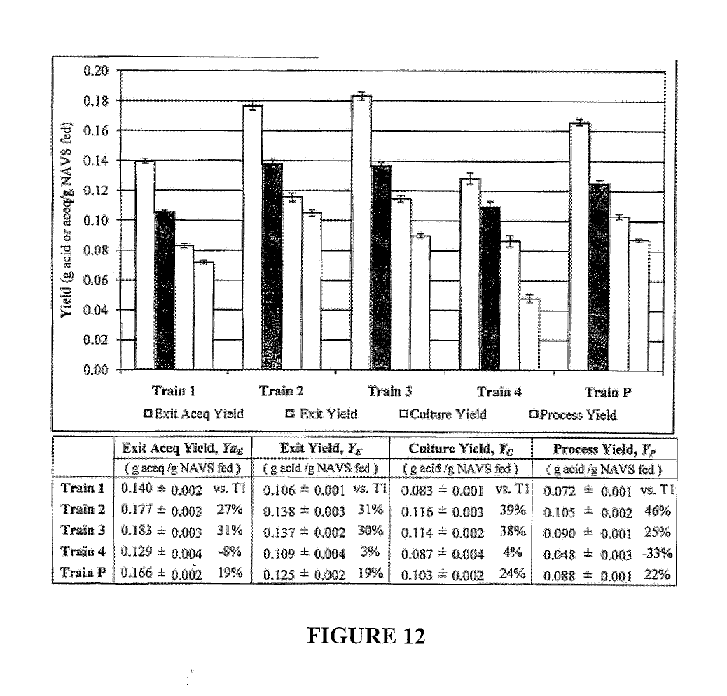

The exit, culture, and process yields were greatly influenced by the nutrient loading pattern (FIG. 12). The exit yield Y_{E} includes the acid in the product transfer liquid, waste transfer solids, and liquid samples taken from F2-F4. The exit aceq yield for Trains 1, 2, 3, 4, and P were 0.140, 0.177, 0.183, 0.129, and 0.166 g aceq/g NAVS fed, respectively. Trains 2, 3, and P had exit aceq yields higher than Train 1 by 27%, 31%, and 19%, respectively. Trains 2 and 3 had statistically identical exit yields (0.138 and 0.137 g acid produced NAYS fed, respectively) with Trains 1, 4, and P having yields of 0.106, 0.109, and 0.125 g acid/g NAYS fed, respectively. Trains 2, 3, 4, and P had exit yields higher than the traditional nutrient addition method (Train 1) by 31%, 30%, 3%, and 19%, respectively. The culture yield Y_{C} represents the acid produced by the microbial cultures, which is equal to the exit yield minus the feed yield. The culture yield Y_{C} for trains 1, 2, 3, 4, and P were 0.083, 0.116, 0.114, 0.087, and 0.103 g acid produced/g NAVS fed, respectively. Trains 2, 3, 4, and P had higher culture yields than Train 1 by 39%, 38%, 4%, and 24%, respectively.

The process yield Yp which quantifies only the acid in the product transfer liquid (L₁) but not the acids in the waste transfer solids (S₄). The process yield is of interest because it quantifies the net yield of acid that is sent downstream for concentration and further processing. In a commercial operation, recovering acids from waste transfer solids requires a countercurrent wash. Because the recovered acid is dilute, it will be returned to Fermenter F4. The liquid flows counter currently relative to the solids, so the recovered acids eventually exit Fermenter F1 and become part of the product transfer liquid (L₁), thus increasing the process yield. In this experiment, no steps were taken to recover acid in the waste transfer solids (S4) and return it to the fermentation; thus, the reported process yields represent the lower process yield limit. The process yield for Trains 1, 2, 3, 4, and P are 0.072, 0.105, 0.090, 0.048, and 0.088 g acid/g NAVS fed, respectively. Trains 2, 3, and P had higher process yields than Train 1 by 46%, 25%, and 22%, respectively. The exit yield Y_{E} represents all the acid exiting the fermentation. If the acids in the waste transfer solids (S₄) are counter currently washed with 100% recovery and the acids are returned to Fermenter F4 but impose no additional product inhibition, then all the acids will exit in the product transfer liquid (L₁). In this ideal scenario, the exit yield represents the theoretical upper limit of process yield.

The process-exit yield ratio (PE ratio) quantifies the fraction of acid recovered in the product transfer liquid. Or the process-exit yield ratio is Y_{P}/Y_{E}. If all acid is recovered from the waste transfer solids, the PE ratio equals 1. The PE ratio for Trains 1, 2, 3, 4, and P were 0.682, 0.761, 0.658, 0.440, and 0.699, respectively. The PE ratios of Trains 2 and 4 were significantly different than Trains 1, 3, and P; thus, PE ratio depends on the nutrient loading pattern. This behavior results from acid in the nutrient feed, and changes in solid-liquid separation, which is affected by the extent of digestion. Additionally, the PE ratio (without recovery of acid in waste transfer solids) depends on the solid-liquid separation efficiency, and the relative flow rates of solids and liquids.

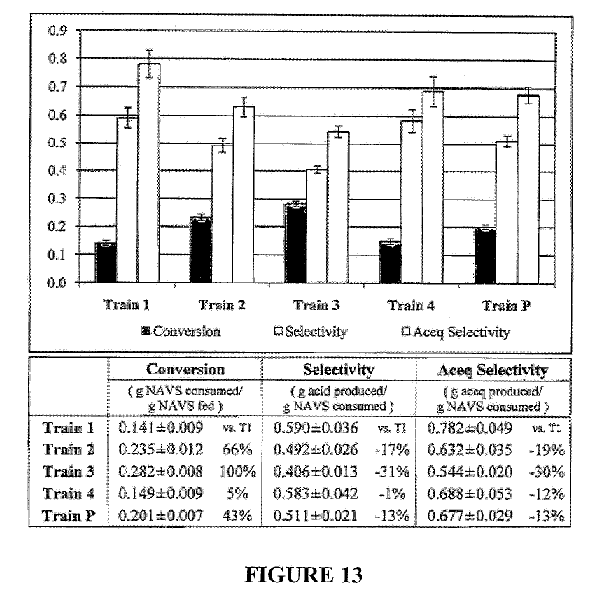

Conversion and selectivity are shown in FIG. 13, and Trains 1, 2, 3, 4, and P had conversions of 0.141, 0.235, 0.282, 0.149, and 0.201 g NAVS consumed/g NAVS fed, respectively. Trains 2, 3, and P had conversions much higher than Train 1 by 66%, 100%, and 43%, respectively. The greatest digestion occurs when both F3 and F4 had near-optimum C/N (25-35 g C_{NA}/g N), which provided nitrogen necessary to digest the most recalcitrant biomass. Train 3 had the highest conversion because it benefits from both near-optimum C/N ratios in F3 and F4, and fresh nutrient feed to F3. Trains 2 had the second highest conversion and benefited from near-optimum CIN in F2-F4. Train P had higher C/N ratios (˜38 g C_{NA}/g N) in the F2-F4, but each fermenter received fresh manure.

Selectivity quantifies the microbial efficiency by reporting the ratio of acid produced in fermentation per mass of NAVS consumed; thus, it is equal to the culture yield divided by conversion (Equation 9). Trains 1, 2, 3, 4, and P had selectivities of 0.590 g, 0.492 g, 0.406 g, 0.583 g, 0.511 g acid produced/g NAVS consumed, respectively. Trains 1 and 4 had the highest selectivities, which were statistically similar. Trains 1, 2, 3, 4, and P had aceq selectivities of 0.782, 0.632, 0.544, 0.688, and 0.677 g acid/g NAVS consumed, respectively.

No train had a selectivity or acid selectivity higher that Train 1. Note, the higher selectivities and aceq selectivities do not correspond with the trains that had the highest yields or highest conversion. This observation supports the hypothesis that nutrient-limited environments increase selectivity because stoichiometric ratios are unavailable to create carbon-rich products (e.g., cells, energy-storage compounds, enzymes) that are non-metabolites.

Nitrogen exists in soluble and insoluble forms traveling in both the transfer solids and transfer liquid streams. Controlling C/N ratios in a countercurrent system is critical to maximizing performance; thus, C/N ratios must be reported to fully understand the context of a fermentation study. The CIN of the feed should be at, or slightly below, the optimum (˜30 g C_{NA}/g N) so that nitrogen is not limiting.

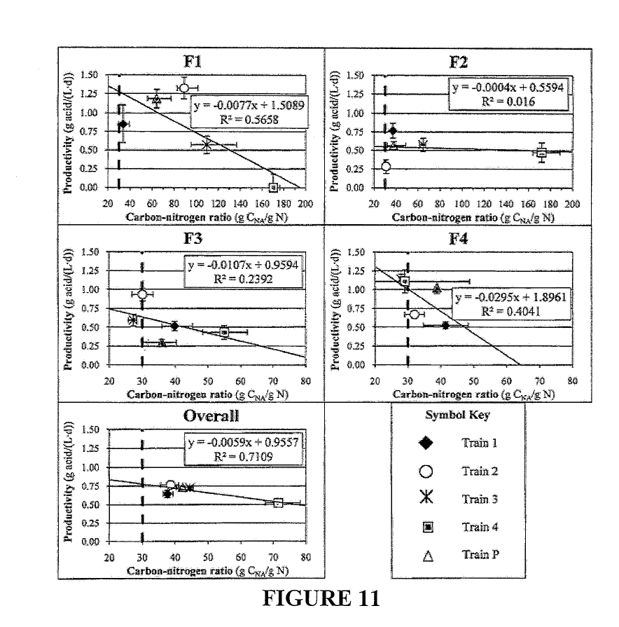

FIGS. 7-12 show patterns that provide insight about an optimum scenario. Acid in the feed reduces the productivity of the receiving fermenter (FIG. 9). Performance improves as the C/N ratio of each fermenter approaches the optimum (30 g C_{NA}/gN) (FIG. 10). It is better to have a few stages close to the optimum C/N ratio (<5 C/N points) rather than all stages near the train's overall C/N ratio (Trains 2 & 3 vs. Train 1). Non-nitrogen nutrients and/or freshness are critical to optimum performance (Train P vs. Train 1). Nutrients are most critical in the latter stages (FIG. 10).

Although, Trains 2 and 3 had the best yields, no single loading pattern should be used generically as an optimum pattern. The nitrogen properties of the feedstocks, the operating parameters, the solid-liquid separation efficiency, and the nutrient loading pattern influence the behavior of nitrogen in a countercurrent fermentation, which dictates performance.

Nutrient feedstocks (e.g., sewage sludge, manure) can contain significant concentrations of organic acids. Characterizing the yield with respect to the feed, exit streams, microbial culture, and product transfer liquid provides greater insight and context to fermentation performance.

### Example 2

As described herein, the MixAlco process is a biorefinery that produces carboxylic acids via anaerobic mixed-acid fermentation. The process uses lignocellulose (e.g., high-yield energy crops, wastes) rather than food crops, which are less productive and more expensive. The carboxylate intermediates are chemically converted into industrial chemicals, solvents, and fuels (e.g., gasoline, alcohols. It has been shown that the MixAlco process can produce gasoline for less than $3/gal; thus, the MixAlco process is an attractive source of renewable energy.

To be economical, the MixAlco process requires high product yields. Previous experiments used a process yield of 0.52 g acid/g NAVS fed. To achieve this, optimization of fermentation is essential. Maintaining sufficient nutrient concentrations and/or proportions is necessary to maximize fermentation performance. Many studies show that carbon-nitrogen ratio greatly influences fermentation yield. Too much or too little nitrogen can limit fermentation performance. Previous mixed-acid fermentations did not quantify or control the carbon-nitrogen ratio (C/N ratio); thus, these fermentations may have been hindered because of excess or limiting nutrients.

Countercurrent fermentation allows for both high product concentrations and high conversions. Nitrogen exists in both soluble and insoluble forms; thus, it travels with both the transfer solid and transfer liquid streams. Nutrient contacting patterns that produced near-optimal carbon-nitrogen (C/N) ratios in each stage of a four-staged countercurrent fermentation dramatically improved yield and conversion. Greater improvements in yield are projected if optimal C/N ratios could be maintained in all stages. To control an optimal C/N profile, a model is needed to describe the behavior and factors that influence nitrogen flow in a countercurrent fermentation. Additionally, a model will provide a tool to evaluate experiments for nutrient limitations, minimize nutrient costs by maximizing use, and understand the influence of model inputs on nitrogen behavior.

Mixed-acid fermentations require both carbohydrate and nutrient components. The carbohydrate component is the primary substrate for acid production, and is loaded to F1; therefore, only the nutrient feed point(s) can be controlled. A nitrogen model is needed that describes both the physical flow of nitrogen in the solid and liquid phases and the flux of nitrogen between these phases. This model develops a mass-balance-based segregated-nitrogen model in which the nitrogen in the solid and liquid phases are segregated and do not influence each other; thus, the difference between modeled and measured nitrogen concentrations is the solid-liquid nitrogen flux.

This model contains the following assumptions: nitrogen is segregated; soluble nitrogen remains soluble and insoluble remains insoluble; nitrogen lost/gained to gaseous phase is negligible; system is at steady state; ideal mixing in each stage; within a stage, the liquid-phase nitrogen concentration is uniform; thus, the concentration of nitrogen in the free liquid and liquid absorbed in the transfer solids are identical; the solid-phase nitrogen concentration is uniform; and nitrogen reactions within a single phase do not influence the nitrogen flow behavior.

Table 1 lists the feedstock properties. FIG. 14 shows the inputs and outputs for the segregated nitrogen model. The inputs may be categorized into four groups: feedstock properties; nutrient feed strategy (Nᵢ); operating parameters, which dictate the feed rates (S₀ and L₅), the size of the fermentation (Fᵢ), and concentration of solids in each stage (1−M_{Fi}); and the solid-liquid separation efficiency, which dictates the moisture contents of the transfer solids (M_{Si}) and liquor (M_{Li}). The solid-liquid separation efficiency depends on the equipment used (centrifuge, screwpress, vacuum filter, etc.) and the degree of digestion of the fermentation solids. Because M_{Si} and M_{Li} are externally influenced, they are considered inputs that must be measured or estimated from other fermentation data.

Five four-bottle fermentation trains each with a different nutrient contacting pattern (FIG. 7), wherein each train was fed a 4:1 ratio (w/w, dry basis) of office-paper and fresh (wet) chicken manure. Each train produced a different nitrogen concentration profile, which were used to determine the validity of the segregated-nitrogen model. Table 2 summarizes the input parameters used for Trains 1, 2, 3, 4, and P.

Methods In this model, two prediction methods are used. FIG. 14 shows the inputs and outputs for both Methods 1 and 2. Method 1 assumes the stream flowrates (Sᵢ and Lᵢ) are unknown, as would occur when designing a fermentation system. It estimates them with an inert-solids material balance. Once the stream flowrates are determined, the values are input into the segregated-nitrogen model to determine the nitrogen parameters of the system. Method 2 assumes stream flowrates are known, which would occur when analyzing an operating fermentation. In this case, measured stream flowrates are input directly into the inert nitrogen model, so the mass balances are not required. The equations are previously presented hereinabove.

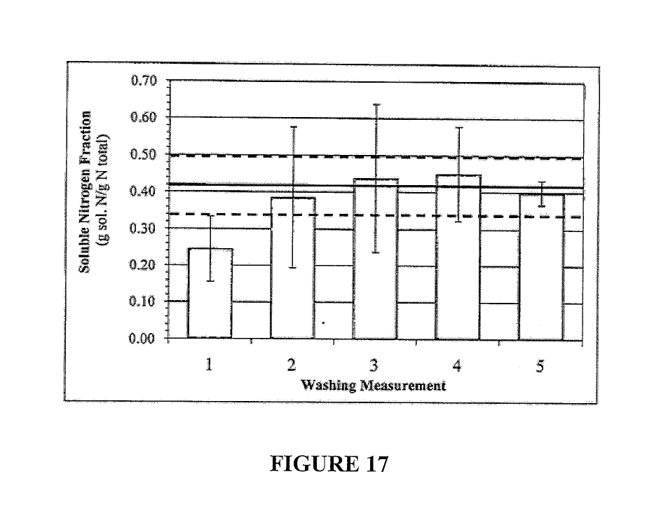

The desired unknowns are v_{Xi} and nₛᵢ. In each term these quantities are part of the compound variables vₓᵢnₓᵢ and vₓᵢ(1−nₓᵢ), which are solved in the system of equations shown in FIG. 17. From these compound variables, vₓᵢ and nₓᵢ may be calculated. Once the stream nitrogen properties (vₛᵢ, nₛᵢ, v_{Li} and n_{Li}) have been determined, they can be used to determine the nitrogen properties of the bulk biomass (v_{Fi}, and n_{Fi}) in each stage. Carbon, nitrogen, and moisture contents were measured according to procedures described herein previously.

TABLE 2

| | | Train 1 | | Train 2 | | Train 3 | | Train 4 | | Train P | |
|---|---|---|---|---|---|---|---|---|---|---|---|
| | Stream/ | Flowrate | M_{XI} | Flowrate | M_{XI} | Flowrate | M_{XI} | Flowrate | M_{XI} | Flowrate | M_{XI} |
| | Stage | (g/T) | (g/100 g)** | (g/T)* | (g/100 g)** | (g/T)* | (g/100 g)** | (g/T)* | (g/100 g)** | (g/T)* | (g/100 g)** |
| Inlet | S₀ | 35.0 | 0.070 | 35.0 | 0.070 | 35.0 | 0.070 | 35.0 | 0.070 | 35.0 | 0.070 |
| Streams | N₁ | 24.0 | 0.660 | 0.0 | 0.660 | 0.0 | 0.660 | 0.0 | 0.660 | 6.0 | 0.660 |
| | N₂ | 0.0 | 0.660 | 24.0 | 0.660 | 0.0 | 0.660 | 0.0 | 0.660 | 6.0 | 0.660 |
| | N₃ | 0.0 | 0.660 | 0.0 | 0.660 | 24.0 | 0.660 | 0.0 | 0.660 | 6.0 | 0.660 |
| | N₄ | 0.0 | 0.660 | 0.0 | 0.660 | 0.0 | 0.660 | 24.0 | 0.660 | 6.0 | 0.660 |
| | L₅ | 300.0 | 1.000 | 300 | 1.000 | 300.0 | 1.000 | 300.0 | 1.000 | 300.0 | 1.000 |
| Transfer | L₁ | 112.4 | 0.980 | 162.6 | 0.980 | 159.6 | 0.980 | 118.2 | 0.980 | 141.2 | 0.980 |
| Streams | S₁ | 180.3 | 0.788 | 177.9 | 0.827 | 166.5 | 0.829 | 164.8 | 0.790 | 176.3 | 0.815 |
| | L₂ | 244.2 | 0.980 | 315.3 | 0.980 | 298.7 | 0.980 | 260.4 | 0.980 | 284.1 | 0.980 |
| | S₂ | 220.5 | 0.849 | 243.6 | 0.847 | 208.1 | 0.854 | 206.7 | 0.858 | 224.4 | 0.853 |
| | L₃ | 292.4 | 0.980 | 366.9 | 0.980 | 348.0 | 0.980 | 311.8 | 0.980 | 333.5 | 0.980 |
| | S₃ | 223.6 | 0.841 | 192.8 | 0.838 | 208.6 | 0.841 | 229.0 | 0.853 | 207.2 | 0.812 |
| | L₄ | 304.3 | 0.980 | 326.5 | 0.980 | 337.3 | 0.980 | 343.0 | 0.980 | 323.7 | 0.980 |
| | S₄ | 209.1 | 0.835 | 159.4 | 0.825 | 162.5 | 0.842 | 193.7 | 0.862 | 180.3 | 0.851 |
| Stages | F₁ | | 0.849 | | 0.891 | | 0.891 | | 0.891 | | 0.880 |
| | F₂ | | 0.920 | | 0.929 | | 0.930 | | 0.930 | | 0.928 |
| | F₃ | | 0.929 | | 0.942 | | 0.938 | | 0.938 | | 0.925 |
| | F₄ | | 0.922 | | 0.935 | | 0.945 | | 0.945 | | 0.945 |

*T = transfer (~56 h)
**wet basis

To determine the soluble nitrogen fraction η, which is a required parameter for the segregated-nitrogen model five fresh (wet) chicken manure samples, were analyzed. To ensure that all the soluble nitrogen was extracted, each sample was washed a specified number of times. Sample 1 was washed once; Sample 2 was washed twice; and so forth. To perform a washing, 30 g of wet manure was placed into a 1-L centrifuge bottle. For each wash, 500 mL of distilled water was added. The capped bottle was shaken for 10 minutes. The mixture was centrifuged at 4000 rpm for 10 minutes. The liquid was decanted and poured into a single container and combined with liquid from successive washes. The masses of the total collected liquid and remaining cake were measured. Samples of each were analyzed for carbon and nitrogen content (% w/w). To determine the amount of soluble nitrogen held by the solids, the moisture content of the cake was measured. The soluble nitrogen fraction q was calculated by dividing the nitrogen mass in the liquid, including the moisture in the cake, by the nitrogen mass in the original sample.

### Example 2

### Results

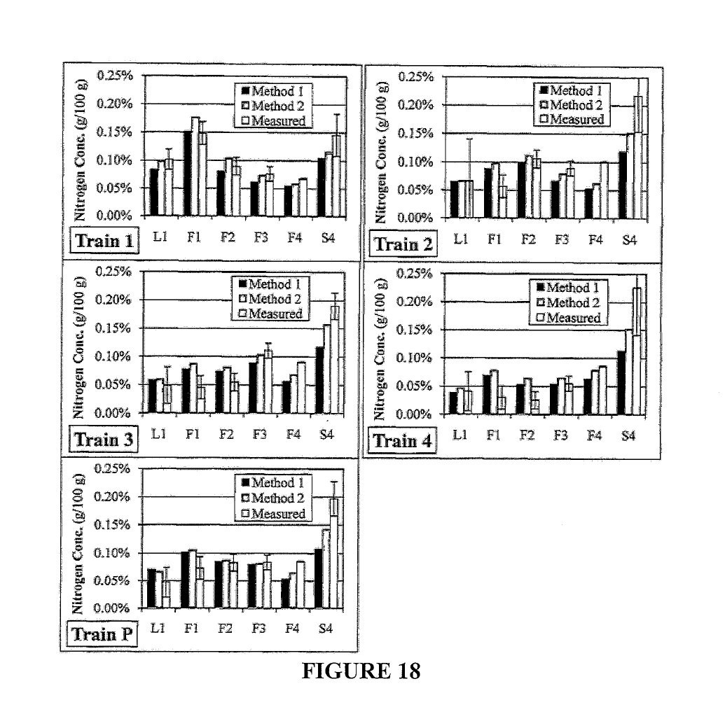

The soluble nitrogen fraction was measured using five samples, each sample receiving a different number of wash cycles. The measured results are shown in FIG. 18. For Sample 1, n was much lower (0.245) than Samples 2-5 (0.385, 0.438, 0.450, 0.400, respectively), indicating not all soluble nitrogen had dissolved in Sample 1. An average value (0.419±0.08) was calculated from Samples 2-5.

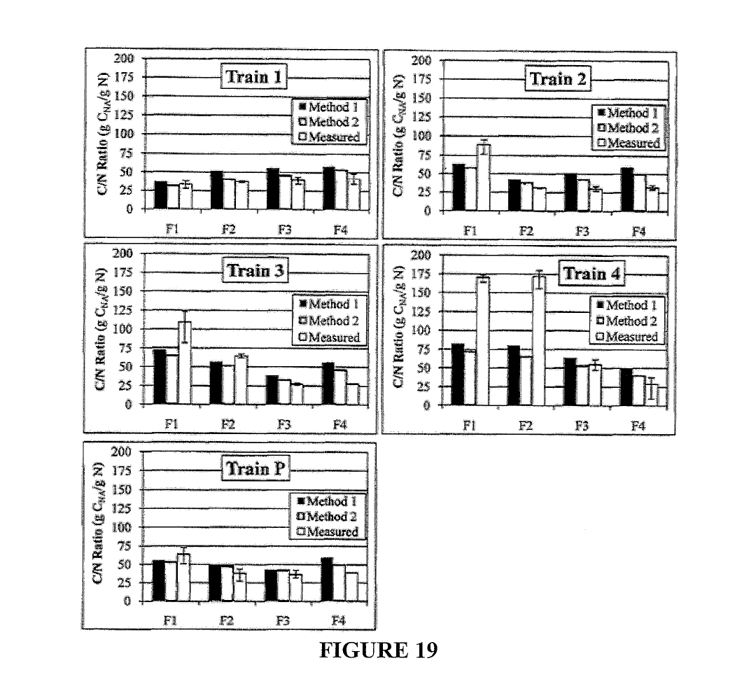

FIG. 19 compares the predicted and measured nitrogen profiles for Trains 1, 2, 3, 4, and P. Both Methods 1 and 2 approximated the measured values. In many cases, the predicted nitrogen concentration is within the measured range. Method 2 is more accurate that Method 1; however, both methods give similar results. For Method 1, the average absolute percent error between measured and predicted nitrogen concentrations for Trains 1, 2, 3, 4, and P were 16% 30%, 37%, 53%, 30%, respectively. For Method 2, the average absolute percent error between measured and predicted nitrogen concentrations for Trains 1, 2, 3, 4, and P were 13%, 26%, 35%, 64%, 24%, respectively. Because conversion has a negative effect on solid stream flowrates, the discrepancy between Method 1 and 2 will increase with conversion; thus, more error may be observed with Method 1 as the volatile solids loading rate (VSLR) and liquid retention (LRT) time decrease, which increases conversion.

The trends of both Method 1 and 2 match the measured profile trends. For Trains 1, 2, 3, and 4, the nutrient-fed fermenter had the highest measured nitrogen concentration. Except for Train 4, both Methods 1 and 2 captured this peak. For all five trains, the measured nitrogen content of the waste transfer solids (S₄) is much greater than the product transfer liquid (L₁). This trend is true for all five trains and is captured by both Methods 1 and 2. Reasonable agreement between predicted and measured shows the segregated-nitrogen model captures basic behavior.

FIG. 19 shows the predicted and measured C/N profiles. Because the non-acid carbon content profile is not sensitive to nutrient feed strategy, the average non-acid carbon content profile of the five trains was used to predict C/N profile. The predicted C/Ns of F1 and F2 of Train 4 had the greatest error; however, Train 4 also had the worst performance of the five trains and was not an optimal nutrient feeding strategy. Except for a few fermenters, the predicted C/N profiles of the better-performing trains (Trains 2, 3, and P) were within 25 C/N points of the measured value; thus, the segregated-nitrogen model is useful for estimating C/N profiles. Because Trains 2, 3, and P approximate the optimal scenario, the discrepancy between the measured and predicted nitrogen profiles (FIG. 10) of these trains indicates the expected discrepancy of an optimal nutrient feeding strategy, which will be a linear combination of Trains 1, 2, 3, and 4.

FIG. 10 shows absolute error (measured minus predicted) profiles for Trains 1, 2, 3, 4, and P, which have a consistent trend among all five trains. In all cases, the measured nitrogen concentration in the product transfer liquid (L₁) and first stages (typically F1 and F2) is less than the predictions. Conversely, the measured concentration in the latter stages (typically F3 and F4) and waste transfer solids (S₄) is greater than the predictions. This diagonal-right error trend can be explained as follows: (1) experimental error, (2) nitrogen lost as gas, and/or (3) reaction between soluble and insoluble forms.

Experimental error analysis shows input stream flowrates and moisture contents were measured accurately. Further, in a sensitivity analysis in which these values were changed within the error bounds, the diagonal-right trend remained. The nitrogen properties (v and n) of the feed are less accurate. In a sensitivity analysis in which these values were changed within the error bounds, the trend does not change; therefore, experimental error does not account for the diagonal-right error trend. Nitrogen lost as gas, was considered, but because the pH was always below 7, significant loss of nitrogen as ammonia gas is unlikely. Because the fermentation is a reducing environment, nitrogen could not be lost as an oxidized species (e.g., NO₂), so significant loss to gaseous nitrogen is not reasonable. Further, if gaseous nitrogen loss were significant, it would only contribute a negative error profile because the measured nitrogen concentrations would be less than the prediction, which is inconsistent with the diagonal-right error trend.

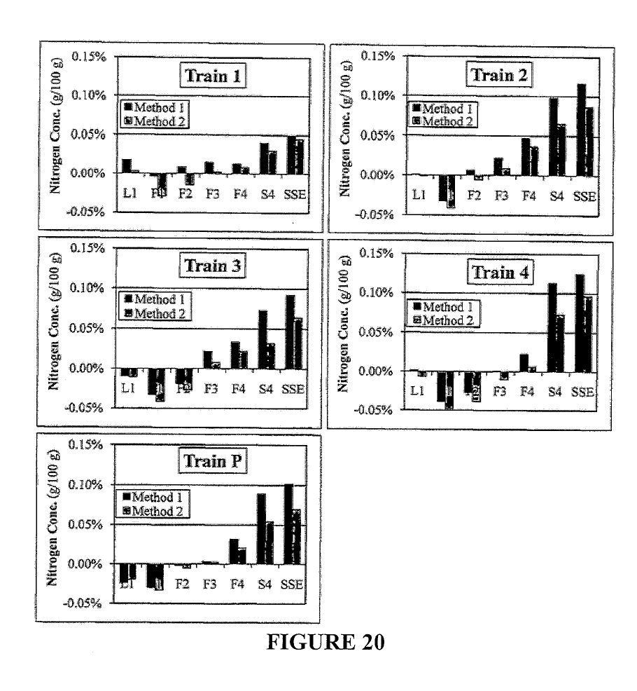

Reaction between soluble and insoluble forms was a core assumption of the model: that soluble and insoluble nitrogen are segregated such that soluble and insoluble nitrogen do not interchange. Violation of this assumption is the most logical explanation. A net reaction flux from soluble to insoluble nitrogen explains the observed diagonal-right error trend, which is consistent with microorganisms metabolizing soluble nitrogen to form cells and insoluble proteins, such as enzymes. The predictions overstate the nitrogen concentration in L₁, F1, and F2 because soluble nitrogen is converted to insoluble nitrogen, which reversed direction leaving these streams and stages with less nitrogen than predicted. Conversely, the predictions understate nitrogen concentrations in F3, F4, and S₄ because the created insoluble nitrogen accumulates in these latter stages. If the net nitrogen flux was from insoluble to soluble, the error profile would flip-flop (diagonal left), which is not observed in FIG. 20.

The sum of squared errors (SSE) measures the cumulative error between the measured and predicted profiles. As a trend, the SSE increases as the feed point moves from F1 to F4. When nutrient is feed to F1, a large fraction of the soluble nitrogen is washed out with the product transfer liquid (L₁); thus, there is less soluble nitrogen to be converted to insoluble-forms, thereby reducing SSE. By contrast, when nutrient is feed to F4, the soluble nitrogen travels with the product transfer liquid and has the most time to convert to insoluble forms and reverse its migration, which increases SSE. The exception to this trend is Train 2, which has a much larger SSE than Train 3.

Assuming all error is caused by nitrogen reaction flux, SSE is a gauge of the flux magnitude. Train 2 had a near-optimal measured C/N in F2-F4, C/Ns equal ˜30 g C_{NA}/gN. Because of its near-optimal C/N profile, Train 2 produced the highest acid yields of the five trains. These observations reinforce the hypothesis that providing optimal nutrients increases the production of cells and hydrolysis enzymes, which increases the production of metabolites (carboxylic acids).

The following explains how the model may be used to determine the optimal nutrient feeding. In a spreadsheet, the system of equations for nitrogen material balances (FIG. 6) was constructed using the segregated-nitrogen model input parameters for Train 2 (Table 3). The system of equations can be solved using “MMULT” and “MINVERSE” functions in I Microsoft Excel. The carbon content profile was assumed to be equal to the average carbon I content profile of Trains 1, 2, 3, 4, and P; the carbon content of Fermenters 1 4 was 0.057, 0.042, 0.035, and 0.032 g C_{NA}/gN wet biomass, respectively. In the spreadsheet, the C/N ratio profile was calculated from the assumed carbon content profile, and the model-determined nitrogen content profile. To determine the nutrient feeding strategy (i.e., optimal N₁, N₂, N₃, and N₄) that would achieve an optimal C/N profile of 30 g C_{NA}/gN, the sum of squared errors was calculated between the calculated profile and the optimal profile. Then, using the “Solver” tool in Microsoft Excel the sum of squared errors was set to zero by changing the values of, N₂, N₃, and N₄.

TABLE 3

| | | Fermentation Train | | | | | |
|---|---|---|---|---|---|---|---|
| | | 1 | 2 | 3 | 4 | P | AVG |
| Controllable | Temperature (° C.) | 40 | 40 | 40 | 40 | 40 | 40 |
| | Frequency (T)* | 3 per week; every 56 h | | | | | |
| | NAVS_{feed} rate (paper & manure) (g VS/T)* | 30.4 | 30.4 | 30.4 | 30.4 | 30.4 | 30.4 |
| | Liquid feed rate (L₅) (mL/T)* | 300 | 300 | 300 | 300 | 300 | 300 |
| | Solid-cake-plus-bottle-weight set point, F1 (g) | 200 | 200 | 200 | 200 | 200 | 200 |
| | Solid-cake-plus-bottle-weight set point, F2-F4 (g) | 300 | 300 | 300 | 300 | 300 | 300 |
| | Centrifuge liquid retained in F1-F4 (mL) | 0 | 0 | 0 | 0 | 0 | 0 |
| | Methane inhibitor (μL/T)* | 80 | 80 | 80 | 80 | 80 | 80 |
| Normalized | VSLR (g NAVS/(L_{IIq} · d)) | 7.5 | 6.7 | 6.8 | 7.1 | 7.0 | 7.0 |
| | LRT (d) | 13.6 | 15.2 | 15.0 | 14.2 | 14.6 | 14.5 |
| | Avg. SC (g NAVS/L_{IIq}) | 57 | 49 | 48 | 55 | 53 | 52 |
| | TLV (L) | 1.75 | 1.96 | 1.93 | 1.83 | 1.87 | 1.87 |

*T = transfer (~56 h)

The optimal nutrient loading rates for N1, N₂, N₃, and N₄ was 21.3, 12.7, 2.5, and 8.8 g wet chicken manure/transfer. From this, example it is shown that (1) the optimal nutrient loading pattern is a linear combination of Trains 1, 2, 3, and 4 (not all nutrient feed to a single fermenter), and (2) the C/N ratio of the feed (35.0 g paper, and 45.3 g wet chicken manure; Table 1) is 28.1 g C_{NA}/gN, which is less than the target C/N ratio, indicating excess nitrogen must be feed to compensate for premature nitrogen loss in the product liquid and waste solid transfer streams. The U-shaped nutrient loading pattern (i.e., greater nutrient feed in F1 and F4 than F2 and F3, respectively) is unexpected and counter intuitive to the results; thus, highlighting the necessity of nutrient transport models for fermentation optimization.

In these models, Nitrogen is a critical element that greatly influences fermentation performance. The segregated-nitrogen model reasonably approximates the measured nitrogen concentration profiles and captures the basic behavior of nitrogen flow in a countercurrent staged fermentation. Therefore, the segregated-nitrogen model may be used to estimate nutrient feeding strategies to achieve an optimal C/N profile, and mathematically understand the influence of input parameters on nitrogen flow. The discrepancies between the model and the data quantify the soluble-insoluble nitrogen reaction flux, and can be used to create a reaction-based model. The data in this paper clearly show a net reaction flux from soluble to insoluble nitrogen; however, this may not be true in general. To improve the segregated-nitrogen model, future research should focus on characterizing and modeling the soluble-insoluble reaction flux.

Method 2, which uses measured stream flows, more accurately predicts measured nitrogen concentration profiles. If stream flow rates are unknown, Method 1, which estimates the stream flows, may be used to estimate nitrogen profiles. The application of this model is not limited to four-stage countercurrent systems and can be adapted to model n-staged systems, as well as to systems with recycle loops. Further, analogous mass-balanced based models could be developed for other critical elements and nutrients (e.g., P and Fe).

The embodiments described and the examples provided herein are exemplary only, and are not intended to be limiting. Many variations and modifications of the invention disclosed herein are possible and are within the scope of the invention. Accordingly, the scope of protection is not limited by the description and examples set out above, but the scope is only limited by the claims which follow, that scope including all equivalents of the subject matter of the claims.

We claim:

1. A method for fermenting biomass comprising:
(a) fermenting biomass in a first fermenter to form digested biomass and a first fermentation broth;
(b) introducing the digested biomass from the first fermenter to a second fermenter having a second fermentation broth; and
(c) introducing a nutrient to at least one of the fermenters.

2. The method of claim 1, comprising prior to step (c):
detecting a property of the fermentation broth in each of the fermenters; and
analyzing the property of the fermentation broth in each of the fermenters.

3. The method of claim 2, wherein the detecting comprises measuring a concentration of the nutrient in the first fermenter broth and the second fermenter broth, and the analyzing comprises determining the difference in the concentration of the nutrient in the first fermenter broth and the second fermenter broth.

4. The method of claim 2, wherein the detecting comprises measuring a concentration of a fermentation product in the first fermenter broth and the second fermenter broth, and the analyzing comprises determining the difference in the concentration of the fermentation product in the first fermenter broth and the second fermentation broth.

5. The method of claim 4, wherein the fermentation product comprises a carboxylate product.

6. The method of claim 1, wherein the nutrient comprises undigested biomass.

7. The method of claim 1, wherein the nutrient comprises essential components for life processes.

8. A method for fermenting biomass comprising:
(a) fermenting biomass in a first fermenter to form digested biomass and a first fermentation broth;
(b) introducing the digested biomass from the first fermenter to a second fermenter having a second fermentation broth; and
(c) introducing a carbon source to at least one of the fermenters.

9. The method of claim 8, comprising prior to step (c):
detecting a property of the fermentation broth in each of the fermenters; and
analyzing the property of the fermentation broth in each of the fermenters.

10. The method of claim 9, wherein the detecting comprises measuring a concentration of the carbon source in the first fermenter broth and the second fermenter broth, and the analyzing comprises determining the difference in the concentration of the carbon source in the first fermenter broth and the second fermenter broth.

11. The method of claim 9, wherein the detecting comprises measuring a concentration of a fermentation product in the first fermenter broth and the second fermenter broth, and the analyzing comprises determining the difference in the concentration of the fermentation product in the first fermenter broth and the second fermentation broth.

12. The method of claim 8, wherein the carbon source comprises undigested biomass.

13. The method of claim 8, wherein the carbon source comprises any biologically available carbon source for essential life processes.

14. A method for fermenting biomass comprising:
(a) fermenting biomass in a first fermenter to form digested biomass and a first fermentation broth;
(b) introducing the digested biomass from the first fermenter to a second fermenter having a second fermentation broth; and
(c) introducing a nutrient and a carbon source to at least one of the fermenters.

15. The method of claim 14, comprising prior to step (c):
detecting at least one property of the fermentation broth in each of the fermenters; and
analyzing at least one property of the fermentation broth in each of the fermenters.

16. The method of claim 15, wherein the detecting comprises measuring a concentration of the fermentation product in the first fermenter broth and the second fermenter broth, and the analyzing comprises determining the difference in the concentration of fermentation product in the first fermenter broth and the second fermenter broth.

17. The method of claim 15, wherein the detecting comprises measuring a concentration of the nutrient in the first fermenter broth and the second fermenter broth, and the analyzing comprises determining the difference in the concentration of the nutrient in the first fermenter broth and the second fermentation broth.

18. The method of claim 15, wherein the detecting comprises measuring a concentration of the carbon source in the first fermenter broth and the second fermenter broth, and the analyzing comprises determining the difference in the concentration of the carbon source in the first fermenter broth and the second fermentation broth.

19. The method of claim 15, wherein the detecting measuring a concentration of the nutrient in the first fermenter broth and the second fermenter broth and measuring a concentration of the carbon source in the first fermenter broth and the second fermenter broth, and the analyzing comprises determining the difference in ratio of the concentration of the nutrient to the concentration of the carbon source in the first fermenter broth and the second fermentation broth

20. The method of claim 14, wherein the nutrient comprises introducing undigested biomass.

21. The method of claim 14, comprising introducing the nutrient to the first fermenter and introducing the carbon source to the second fermenter.

22. The method of claim 14, comprising introducing the nutrient to the second fermenter and introducing the carbon source to the first fermenter.

23. The method of claim 14, comprising introducing the nutrient and the carbon source at a predetermined ratio.

24. A method for fermenting biomass comprising:
(a) fermenting a first biomass in a first fermenter to form a first digested biomass and a first fermentation broth;
(b) fermenting a second biomass in a second fermenter to form a second digested biomass and a second fermentation broth;
(c) fermenting a third biomass in a third fermenter to form third digested biomass and a third fermentation broth;
(d) detecting at least one property of each of the fermentation broths for analysis; and
(e) introducing the first digested biomass to the second fermentation broth in the second fermenter;
(f) introducing the first fermentation broth to the third digested biomass in the third fermenter; and
(g) introducing the third fermentation broth to the second digested biomass.

25. The method of claim 24, wherein the first biomass comprises undigested biomass; and
the second biomass and third biomass comprise at least partially digested biomass.

26. The method of claim 24, wherein (d) further comprises:
comparing the least one detected property of each fermentation broths against each other and against a predetermined optimization of the at least one detected property; and
determining which fermentation broth to introduce to which digested biomass.

27. The method of claim 26, wherein the at least one property may comprise one chosen from the group consisting of pH, nutrient concentration, carbon source concentration, nutrient concentration to carbon concentration ratio, and combinations thereof.

28. A fermenter system comprising;
a plurality of fermenters, having a first fermenter, a last fermenter, and at least one intermediate fermenter, wherein the first fermenter comprises the inlet for biomass, and the last fermenter comprises the inlet for fermentation broth;
a plurality of conduits disposed between each of the plurality of the fermenters;
a sensor system, having a sensor positioned in each of the plurality of fermenters; and
a nutrient supply, fluidly connected with each of the plurality of fermenters.

29. The fermenter system of claim 28, wherein the plurality of conduits comprise:
a first portion of the plurality of conduits configured to convey biomass between each of the plurality of fermenters; and
a second portion of the plurality of conduits configured to convey fermentation broth between each of the plurality of fermenters.

30. The fermenter system of claim 28, further comprising a control system, wherein in response to the sensor system, the control system is configured to control flow through the plurality of conduits and the nutrient supply.

31. The fermenter system of claim 28 wherein the nutrient supply comprises at least one selected from the group consisting of: undigested biomass, a nutrient-rich supply, a carbon-rich supply, and combinations thereof.
